(19) United States
(12) Patent Application Publication
(10) Pub. No.: US 2024/0165190 A1
(43) Pub. Date: May 23, 2024

(54) A HERBO-MINERAL METALLIC PHARMACEUTICAL KIT

(71) Applicant: Sadanand Prabhakar SARDESHMUKH, Pune (IN)

(72) Inventors: Sadanand Prabhakar SARDESHMUKH, Pune (IN); Vineeta Vasant DESHMUKH, Pune (IN)

(21) Appl. No.: 18/281,545

(22) PCT Filed: Mar. 11, 2022
(86) PCT No.: PCT/IB2022/052192

§ 371 Date: Sep. 11, 2023

(51) Int. Cl. A61K 36/8965; A61K 36/35; A61K 36/59; A61K 36/61; A61K 36/67; A61K 47/36; A61P 35/04

(52) U.S. Cl. CPC A61K 36/8965; A61K 36/35; A61K 36/59; A61K 36/61; A61K 36/67; A61K 47/36; A61P 35/04

(57) ABSTRACT

The present disclosure relates to a herbo-mineral metallic pharmaceutical kit for delaying/controlling recurrence and/or metastasis of cancer in Triple Negative Breast Cancer (TNBC) patients. The herbo-mineral metallic pharmaceutical kit comprises a first container containing Suvarna Bhasmadi Vati (SBV), a second container containing Mouktikyukta Kamdudha Vati (MKV), a third container containing Shatavari Vati (SV), a fourth container containing Arogyavardhini Vati (AVV), a fifth container containing Triphala Guggul Vati (TGV) and a sixth container containing jatamansi Oil (JMO). The herbo mineral metallic pharmaceutical kit assists in improving immunomodulatory response and reducing oxidative stress in TNBC patients.

### FIELD

The present disclosure relates to a herbo-mineral metallic pharmaceutical kit. Particularly, the present disclosure relates to a herbo-mineral metallic pharmaceutical kit for Triple Negative Breast Cancer (TNBC) patients.

### ABBREVIATIONS

Hb: Hemoglobin
WBC: White blood cells.
SGOT: Serum glutamic oxaloacetic transaminase.
SGPT: Serum glutamic pyruvic transaminase.
CRP: C-Reactive Protein
RSR: Relative Survival Rate
QoL: Quality of Life
QLQ C 30: Quality of Life Questionnaire
IL: Interleukin
SOD: Superoxide Dismutase
BRCA1 or BRCA2: BRCA is a human tumor suppressor gene (also known as a caretaker gene) and is responsible for repairing DNA.
TNBC: Triple Negative Breast Cancer is a heterogenous disease in which the three genes—encoding estrogen receptor (ER), progesterone receptor (PR) and HER2/neu Protein do not express and hence it results in negative testing for these receptors.
PET CT: Positron Emission Tomography—Computed Tomography is a type of nuclear medical imaging to diagnose, evaluate or treat variety of diseases.

### Definitions

As used in the present disclosure, the following terms are generally intended to have the meaning as set forth below, except to the extent that the context in which they are used indicate otherwise.

Suvarna Bhasma: The term “Suvarna Bhasma” refers to “incinerated gold” prepared by incinerating gold in accordance with the present disclosure. The term “Suvarna Bhasma” referred in the present disclosure is not the same as used in the Ayurveda. Suvarna refers to 24 carat gold.

Mouktik Bhasma: The term “Mouktik Bhasma” refers to “incinerated pearl”, natural or cultured, prepared by incinerating pearl in accordance with the present disclosure. The term “Mouktik Bhasma” referred in the present disclosure is not the same as used in the Ayurveda.

Shankha Bhasma: The term “Shankha Bhasma” refers to “incinerated Conch shell”. The term “Shankha Bhasma” referred in the present disclosure is not the same as used in the Ayurveda.

Shouktik Bhasma: The term “Shouktik Bhasma” refers to “incinerated empty pearl shell”. The term “Shouktik Bhasma” referred in the present disclosure is not the same as used in the Ayurveda.

Kapardika Bhasma: The term “Kapardika Bhasma” refers to “incinerated Cowries”. The term “Kapardika Bhasma” referred in the present disclosure is not the same as used in the Ayurveda.

Pravala Bhasma: The term “Pravala Bhasma” refers to “incinerated coral”. The term “Pravala Bhasma” referred in the present disclosure is not the same as used in the Ayurveda.

Shudhha Gairik: The term “Gairik” is a natural clay earth pigment which is a mixture of ferric oxide and varying amounts of clay and sand. “Shudhha Gairik” refers to “processed Gairik”, prepared by roasting Gairik in ghee obtained from cow's milk. The term “Gairik” referred in the present disclosure is not the same as used in the Ayurveda.

Guduchi Sattva: The term “Guduchi Sattva” refers to extract comprising mainly starch of Tinospora cordifolia/sinensis/crispa/glabra prepared by alcoholic, hydro-alcoholic or aqueous extraction method. The term “Guduchi Sattva” referred in the present disclosure is not the same as used in the Ayurveda.

Kajjali: The term “Kajjali” refers to “black sulphide of mercury”. The term “Kajjali” referred in the present disclosure is not the same as used in the Ayurveda.

Tamra Bhasma: The term “Tamra Bhasma” refers to “incinerated copper”. The term “Tamra bhasma” referred in the present disclosure is not the same as used in the Ayurveda.

Abhrak Bhasma: The term “Abhrak Bhasma” refers to “incinerated mica”. The term “Abhrak bhasma” referred in the present disclosure is not the same, as used in the Ayurveda.

loha Bhasma: The term “loha Bhasma” refers to “incinerated iron”. The term “loha bhasma” referred in the present disclosure is not the same, as used in the Ayurveda.

Haritaki: The term “Haritaki” refers to “Terminalia chebula”.

Bibhitaki: The term “Bibhitaki” refers to “Terminalia bellerica”.

Amalaki: The term “Amalaki” refers to “Emblica officinalis”.

Shuddha Shilajit: The term refers to purified “Black bitumen or Mineral pitch or asphaltum processed in the decoction of Triphala.

Shuddha Guggul: The term refers to purified “Commiphora mukul or Commiphora wightii or other related Guggulu species processed in decoction of Triphala.

Chitrak: The term “Chitrak” refers to “Plumbago zeylanica” or related species.

Kutaki: The term “Kutaki” refers to “Picrorrhiza kurroa”.

Nimba: The term “Nimba” refers to “Azadirachta indica”.

Pippali: The term “Pippali” refers to “Piper longum”.

Shatavari: The term “Shatavari” refers to “Asparagus racemosus” or related species.

jatamansi: The term “jatamansi” refers to “Nardostachys jatamansi” or related species

Til taila: The term “Til taila” refers to Sesame Oil

Vati: The term “Vati” refers to a method of medicine preparation in which herbs, minerals, and metallic compounds are compressed into tablet form.

Trituration: The term “trituration” refers to either reducing the particle size of a substance or production of a homogeneous material by mixing component materials thoroughly or wet grinding any material with a liquid media like fresh juice or decoction etc.

Karnofsky score: The term “Karnofsky score” refers to the Karnofsky Performance Scale Index allows patients to be classified as to their functional impairment

Symptom score: The term “Symptom score” of QLQ is indicative of symptomatology. Hence, decrease in symptom score represents both decrease in disease related symptoms and adverse effects of conventional treatment.

Function score: The term “Functional score” of QLQ signifies status of routine physical activities. Increase in functional scores represents improvement in QoL.

Global score: The term “Global score of QLQ” represents overall well-being of a patient. Increase in global scores represents improvement in QoL.

Neoadjuvant chemotherapy: The term “Neoadjuvant chemotherapy” refers to medicines that are administered before surgery for the treatment of breast cancer.

Shiropichu: The term “Shiropichu” refers to a type of head massage in which a cotton gauze piece of 5 cm diameter is soaked in oil and kept on the head for a period of 15-20 minutes, after which the oil is squeezed onto the scalp followed by gentle head massage.

Clinical parameters: The term “clinical parameters” refers to the levels of Serum bilirubin (Total), Serum alkaline phosphatase, Serum Creatinine, Serum Urea, C-Reactive Protein, IL1, IL6, IL8 and IL10, SOD, Catalase, Glutathione in a subject.

### BACKGROUND

The background information herein below relates to the present disclosure but is not necessarily a prior art.

Triple Negative Breast Cancer (TNBC) is a heterogeneous disease in which three genes—encoding Estrogen Receptor (ER), Progesterone Receptor (PR) and HER2/neu protein do not express. Hence, the testing of samples of TNBC result in negative testing for these receptors. An estimated one million cases of breast cancer are diagnosed annually worldwide. Among these, 15-20% of women (more than 1,70,000) are diagnosed as triple-negative, while majority of the TNBC patients are found to be young women or women with a mutation in the BRCA1 gene. Prevalence of TNBC in India is considerably higher compared with that seen in western populations, with as many as one in three women with breast cancer could have triple-negative disease.

The pattern of metastatic spread in TNBC is different from that of the other breast cancer subtypes with a higher likelihood of brain and lung involvement and less frequent bone lesions. In addition, this is the tumor subtype with the poorest prognosis between all the breast cancer subtypes. Major challenges in TNBC are its unique molecular profile, aggressive nature, distinct metastatic patterns and lack of targeted therapies. It is known that tumors expressing receptors for estrogen and progesterone generally respond well to hormonal interventions. HER2+ tumors (which overexpress the ERBB2 oncogene) respond effectively when anti-HER2 therapy is used.

However, tumors that lack expression of all three receptors (TNBC) are very aggressive with no current molecular-based targeted therapies available. Moreover, only 20% of TNBC tumors respond well to neo-adjuvant chemotherapy, and those with residual disease after treatment have a significantly worse survival than those of different molecular subtypes. Studies have shown that Triple Negative Breast Cancer (TNBC) is more likely to spread beyond the breast and to recur after treatment. Also, it tends to be higher grade than other types of breast cancer as it usually involves a “basal-like” cell type. The cells resemble the basal cells that line the breast ducts. Basal-like cancers tend to be more aggressive, higher grade cancers, just like triple-negative breast cancers. Additionally, TNBC are also known for an early peak of recurrence between the first and the third year after diagnosis, and very aggressive metastases, which are more likely to occur in viscera particularly in the lungs and brain, and less likely to spread to the bone.

Standard treatment for TNBC is surgery with adjuvant chemotherapy and radiotherapy. As a variation, neoadjuvant chemotherapy is very frequently used in TNBC. Although TNBC patients respond to chemotherapy better than other types of breast cancer, prognosis remains poor due to shortened disease-free interval in the adjuvant and neoadjuvant setting and a more aggressive course in the metastatic setting. As TNBC does not respond to the hormone therapies, treatment options are much limited than those for other types of breast cancer.

The progress in the conventional treatment of TNBC remains an important challenge. This clarifies the need for developing and evaluating new strategies with superior efficacy or compliance in preventing occurrence of cancer and preventing or delaying the onset of second malignancy. This results in increasing cancer free survival with good physical and mental quality of life. In this respect a herbo-mineral metallic pharmaceutical kit may prove to be helpful in controlling/delaying the recurrence, occurrence in second breast or metastasis in other vital organs.

Therefore, there is felt a need to provide a selected herbo-mineral metallic combination in the form of a herbo-mineral metallic pharmaceutical kit that mitigates the afore-stated problems.

### OBJECTS

Some of the objects of the present disclosure, which at least one embodiment herein satisfies, are as follows:

An object of the present disclosure is to ameliorate one or more problems of the prior art or to at least provide a useful alternative.

Another object of the present disclosure is to provide a herbo-mineral metallic pharmaceutical kit.

Yet another object of the present disclosure is to provide a herbo-mineral metallic pharmaceutical kit for delaying/controlling recurrence, occurrence in second breast/metastasis in Triple Negative Breast Cancer patients.

Yet another object of the present disclosure is to provide a herbo-mineral metallic pharmaceutical kit for maintaining/improving clinical parameters of Triple Negative Breast Cancer patients.

Another object of the present disclosure is to provide a herbo-mineral metallic pharmaceutical kit for reducing oxidative stress in Triple Negative Breast Cancer patients.

Yet another object of the present disclosure is to provide a herbo-mineral metallic pharmaceutical kit for improving the immune response in Triple Negative Breast Cancer patients.

Other objects and advantages of the present disclosure will be more apparent from the following description, which is not intended to limit the scope of the present disclosure.

### SUMMARY

The present disclosure provides a herbo-mineral metallic pharmaceutical kit for delaying/controlling the recurrence of cancer or metastasis of cancer in second breast or other vital organs in Triple Negative Breast Cancer patients.

In an aspect, the present disclosure provides a kit comprising a first container containing Suvarna Bhasmadi Vati (SBV) in a solid dosage form in the range of 250 mg to 450 mg, a second container containing Mouktikyukta Kamdudha Vati (MKV) in a solid dosage form in the range of 300 mg to 600 mg, a third container containing Shatavari Vati (SV) in a solid dosage form in the range of 300 mg to 600 mg, a fourth container containing Arogyavardhini Vati (AVV) in a solid dosage form in the range of 300 mg to 600 mg, a fifth container containing Triphala Guggul Vati (TGV) in a solid dosage form in the range of 300 mg to 600 mg and a sixth container contains jatamansi taila (JMO) in the form of medicated oil for external application in sufficient quantity.

The first container containing Suvarna Bhasmadi Vati (SBV) is prepared from Suvarna Bhasma in an amount ranging from 2 wt. % to 7 wt. % of the total weight of the SBV, Mouktik Bhasma in an amount ranging from 20 wt. % to 35 wt. % of the total weight of the SBV, Guduchi Sattva in an amount ranging from 45 wt. % to 60 wt. % of the total weight of the SBV and at least one excipient in an amount ranging from 5 wt. % to 30 wt. % of the total weight of the SBV.

In accordance with an embodiment of the present disclosure, the SBV comprises Suvarna Bhasma in an amount ranging from 3 wt. % to 5 wt. % of the total weight of the SBV, Mouktik Bhasma in an amount ranging from 23 wt. % to 30 wt. % of the total weight of the SBV, Guduchi Sattva in an amount ranging from 48 wt. % to 54 wt. % of the total weight of the SBV, and at least one excipient in an amount ranging from 10 wt. % to 25 wt. % of the total weight of the SBV.

The second container containing Mouktikyukta Kamdudha Vati (MKV) is prepared from Mouktik Bhasma in an amount ranging from 10 wt. % to 14 wt. % of the total weight of the MKV, Shankha Bhasma in an amount ranging from 10 wt. % to 14 wt. % of the total weight of the MKV, Shouktik Bhasma in an amount ranging from 10 wt. % to 14 wt. % of the total weight of the MKV, Kapardik Bhasma in an amount ranging from 10 wt. % to 14 wt. % of the total weight of the MKV, Praval Bhasma in an amount ranging from 10 wt. % to 14 wt. % of the total weight of the MKV, Guduchi Sattva in an amount ranging from 10 wt. % to 14 wt. % of the total weight of the MKV, Shudhha Gairik in an amount ranging from 10 wt. % to 14 wt. % of the total weight of the MKV, and at least one excipient in an amount ranging from 10 wt. % to 30 wt. % of the total weight of the MKV.

The third container containing Shatavari Vati (SV) is prepared from powder obtained from Shatavari in an amount ranging from 75 wt. % to 92 wt. % of the total weight of the Shatavari Vati (SV), and at least one excipient in an amount ranging from 8 wt. % to 25 wt. % of the total weight of the Shatavari Vati (SV).

The fourth container containing Arogyavardhini Vati (AVV) is prepared from Kajjali in an amount ranging from 2.3 wt. % to 2.5 wt. % of the total weight of the AVV, loha Bhasma in an amount ranging from 1.1 wt. % to 1.3 wt. % of the total weight of the AVV, Abhrak Bhasma in an amount ranging from 1.1 wt. % to 1.3 wt. % of the total weight of the AVV, Tamra Bhasma in an amount ranging from 1.1 wt. % to 1.3 wt. % of the total weight of the AVV, Haritaki powder in an amount ranging from 2.3 wt. % to 2.5 wt. % of the total weight of the AVV, Bibhitaki powder in an amount ranging from 2.3 wt. % to 2.5 wt. % of the total weight of the AVV, Amalaki powder in an amount ranging from 2.3 wt. % to 2.5 wt. % of the total weight of the AVV, Shuddha Shilajit in an amount ranging from 3.5 wt. % to 3.7 wt. % of the total weight of the AVV, Shuddha Guggul in an amount ranging from 4.7 wt. % to 4.9 wt. % of the total weight of the AVV, Chitrak powder in an amount ranging from 4.7 wt. % to 4.9 wt. % of the total weight of the AVV, Kutaki powder in an amount ranging from 26.2 wt. % to 26.8 wt. % of the total weight of the AVV, the dry extract from Nimba leaf juice in an amount ranging from 22 wt. % to 22.4 wt. % of the total weight of the AVV, and at least one excipient in an amount ranging from 23.4 wt. % to 26.4 wt. % of the total weight of the AVV.

The fifth container containing Triphala Guggul Vati (TGV) comprises Amalaki powder in an amount ranging from 9.6 wt. % to 11.6 wt. % of the total weight of the TGV, Bibhitaki powder in an amount ranging from 9.6 wt. % to 11.6 wt. % of the total weight of the TGV, Haritaki powder in an amount ranging from 9.6 wt. % to 11.6 wt. % of the total weight of the TGV, Pippali powder in an amount ranging from 9.6 wt. % to 11.6 wt. % of the total weight of the TGV, Shuddha Guggul in an amount ranging from 50 wt. % to 56 wt. % of the total weight of the TGV, and at least one gliding agent in an amount ranging from 3.6 wt. % to 5.6 wt. % of the total weight of the TGV.

The sixth container containing jatamansi taila (JMO) comprises extract of jatamansi in an amount ranging from 0.5 wt. % to 2 wt. % of the total weight of the JMO and Til taila in an amount ranging from 99.5 wt. % to 98 wt. % of the total weight of the JMO. The compositions of SBV, MKV, SV, AVV and TGV are prepared in the form of solid unit dosage form individually selected from the group consisting of tablet, pill, and capsule, while the composition JMO is prepared as medicated oil.

### BRIEF DESCRIPTION OF THE ACCOMPANYING DRAWING

The present disclosure will now be described with the help of the accompanying drawing, in which:

FIG. 1 illustrates a graph representing the effect of the herbo-mineral metallic pharmaceutical composition on haemoglobin in Triple Negative Breast Cancer patients;

FIG. 2 illustrates a graph representing the effect of the herbo-mineral metallic pharmaceutical composition on WBC count in Triple Negative Breast Cancer patients;

FIG. 3 illustrates a graph representing the effect of the herbo-mineral metallic pharmaceutical composition on platelet count in Triple Negative Breast Cancer patients;

FIG. 4 illustrates a graph representing the effect of the herbo-mineral metallic pharmaceutical composition on S. bilirubin in Triple Negative Breast Cancer patients;

FIG. 5 illustrates a graph representing the effect of the herbo-mineral metallic pharmaceutical composition on SGOT in Triple Negative Breast Cancer patients;

FIG. 6 illustrates a graph representing the of effect of the herbo-mineral metallic pharmaceutical composition on SGPT in Triple Negative Breast Cancer patients;

FIG. 7 illustrates a graph representing the effect of the herbo-mineral metallic pharmaceutical composition on S. Alkaline Phosphatase in Triple Negative Breast Cancer patients;

FIG. 8 illustrates a graph representing the effect of the herbo-mineral metallic pharmaceutical composition on S. Creatinine in Triple Negative Breast Cancer patients;

FIG. 9 illustrates a graph representing the effect of the herbo-mineral metallic pharmaceutical composition on S. Urea level in Triple Negative Breast Cancer patients;

FIG. 10 illustrates a graph representing the effect of the herbo-mineral metallic pharmaceutical composition on CA 15.3 level in Triple Negative Breast Cancer patients;

FIG. 11 illustrates a graph representing the effect of the herbo-mineral metallic pharmaceutical composition on CRP level in Triple Negative Breast Cancer patients;

FIG. 12 illustrates a graph representing the effect of the herbo-mineral metallic pharmaceutical composition on IL1β level in Triple Negative Breast Cancer patients;

FIG. 13 illustrates a graph representing the effect of the herbo-mineral metallic pharmaceutical composition on IL6 level in Triple Negative Breast Cancer patients;

FIG. 14 illustrates a graph representing the effect of the herbo-mineral metallic pharmaceutical composition on IL8 level in Triple Negative Breast Cancer patients;

FIG. 15 illustrates a graph representing the effect of the herbo-mineral metallic pharmaceutical composition on IL10 level in Triple Negative Breast Cancer patients;

FIG. 16 illustrates a graph representing the effect of the herbo-mineral metallic pharmaceutical composition on SOD level in Triple Negative Breast Cancer patients;

FIG. 17 illustrates a graph representing the effect of the herbo-mineral metallic pharmaceutical composition on catalase level in Triple Negative Breast Cancer patients;

FIG. 18 illustrates a graph representing the effect of the herbo-mineral metallic pharmaceutical composition on glutathione level in Triple Negative Breast Cancer patients;

FIG. 19 illustrates a graph representing the effect of the herbo-mineral metallic pharmaceutical composition on Karnofsky score and weight in Triple Negative Breast Cancer patients; and

FIG. 20 illustrates a graph representing the effect of the herbo-mineral metallic pharmaceutical composition on Quality of Life (QoL) in Triple Negative Breast Cancer patients.

### DETAILED DESCRIPTION

Embodiments, of the present disclosure, will now be described with reference to the accompanying drawings.

Embodiments are provided so as to thoroughly and fully convey the scope of the present disclosure to the person skilled in the art. Numerous details are set forth, relating to specific components, and methods, to provide a complete understanding of embodiments of the present disclosure. It will be apparent to the person skilled in the art that the details provided in the embodiments should not be construed to limit the scope of the present disclosure. In some embodiments, well-known processes, well-known apparatus structures, and well-known techniques are not described in detail.

The terminology used, in the present disclosure, is only for the purpose of explaining a particular embodiment and such terminology shall not be considered to limit the scope of the present disclosure. As used in the present disclosure, the forms “a,” “an,” and “the” may be intended to include the plural forms as well, unless the context clearly suggests otherwise. The terms “comprises,” “comprising,” “including,” and “having,” are open ended transitional phrases and therefore specify the presence of stated features, integers, steps, operations, elements, modules, units and/or components, but do not forbid the presence or addition of one or more other features, integers, steps, operations, elements, components, and/or groups thereof. The particular order of steps disclosed in the method and process of the present disclosure is not to be construed as necessarily requiring their performance as described or illustrated. It is also to be understood that additional or alternative steps may be employed.

Breast cancer continues to be the second cause of death in women worldwide. TNBC represents approximately 15-20% of all diagnosed breast cancers, which amounts to more than 1,70,000 cases each year. TNBC is usually treated with surgery, chemotherapy and radiation. Chemotherapy remains the standard of care for TNBC because no targeted therapies have been proven to be effective for this subtype. Neoadjuvant chemotherapy i.e. chemotherapy before surgery includes breast-conserving surgery as well as assessing response to systemic therapy. Although TNBC patients respond to chemotherapy better than the other types of breast cancer, prognosis remains poor due to shortened disease-free interval in the adjuvant and neoadjuvant setting and a more aggressive course in the metastatic setting. Even though TNBC doesn't usually respond to hormone therapy treatment, newer medications called poly ADP-ribose polymerase (PARP) inhibitors as well as immunotherapy are sometimes used to treat TNBC. But finding a better treatment for TNBC is a major focus for breast cancer research. Hence, looking at the aggressiveness of TNBC with poor survival, there is an urgent need to explore other non-surgical methods for disease free survival.

The present disclosure provides a herbo-mineral metallic pharmaceutical composition for controlling/delaying the recurrence, occurrence of cancer in second breast or metastasis in other vital organs. The present disclosure provides a selected combination of herbo-mineral metallic pharmaceutical compositions, in the form of a kit.

In an aspect, the present disclosure provides a herbo-mineral metallic pharmaceutical kit for reducing oxidative stress, improving immune status and quality of life and increase disease free survival in TNBC cancer patients.

The kit comprises a first container containing Suvarna Bhasmadi Vati (SBV) in a solid dosage form in an amount in the range of 250 mg to 450 mg, a second container containing Mouktikyukta Kamdudha Vati (MKV) in a solid dosage form in an amount in the range of 300 mg to 600 mg, a third container containing Shatavari Vati (SV) in a solid dosage form in an amount in the range of 300 mg to 600 mg, a fourth container containing Arogyavardhini Vati (AVV) in a solid dosage form in an amount in the range of 300 mg to 600 mg and fifth container containing Triphala Guggul Vati (TGV) in a solid dosage form in an amount in the range of 300 mg to 600 mg.

The rationale for selecting the individual components of the kit and their compositions are as follows:

Suvarna Bhasmadi Vati (SBV) prepared in accordance with the present disclosure contains Suvarna Bhasama which is rejuvenating, possesses detoxifying and immunomodulatory actions.

Mouktik Bhasma has strong anti-inflammatory action and helps to reduce oxidative stress in the body. Guduchi Satva is also rejuvenating, and immunomodulatory. Beneficial effect of Guduchi (Tinospora cordifolia) in breast cancer patients is well-documented.

Mauktikyukta Kamadudha Vati (MKV) is anti-inflammatory and reduces oxidative stress in the body, thus helps to control cancer progression.

Shatavari Vati (SV) boosts functions of breast tissues and acts as immunomodulatory agent. It is also beneficial to reduce oxidative stress.

Arogyavardhini Vati (AVV) detoxifies vital organs in the body including breast. It enhances functions of liver and lung, which are the common sites of metastasis in TNBC. It also improves digestion, metabolism and liver function, which in turn hampers growth of tumor.

Triphala Guggul Vati (TGV) is beneficial to control initiation, progression and metastatization of cancer, especially in Triple Negative Breast Cancer. It also possesses immunomodulatory and anti-inflammatory activity.

jatamansi Oil (JMO) is used for Shiro Pichu (a special type of head massage) which is beneficial to relieve mental stress, a common mental health condition that affects patients with TNBC. It is found that jatamansi oil is significantly effective in relieving symptoms of mental stress namely sleep disturbance, difficulty in concentration, fearfulness, restlessness, irritability, crying spells, fearfulness and sleep disturbances and thus providing relaxation of mind. Further, jatamansi oil is proved to be significantly effective in scores of Zung's Self Rating Depression scale and Zung's Self Rating Anxiety scale in TNBC patients. Thus, the herbo-mineral metallic pharmaceutical kit of present disclosure when administered in the form of combination of polyherbal compositions, acts synergistically in improving immunomodulatory activity and provides a rejuvenation effect. It has anti-inflammatory, anti-tumor, hepatoprotective, antioxidant and detoxifying properties. The polyherbal composition of the kit improves digestion and metabolism; thus improving quality of life and disease free survival in TNBC patients.

By way of example, it is demonstrated that the present kit envisaged in this disclosure is suitable for improving quality of life and increasing disease free survival by preventing recurrence/metastasis in TNBC patients. It is also demonstrated that the survival patterns of patients after administration of different combination of herbo-mineral metallic composition other than present disclosure do not provide the desirable effect.

The Suvarna Bhasmadi Vati (SBV) in the present pharmaceutical composition is prepared from Suvarna Bhasma in an amount ranging from 2 wt. % to 7 wt. % of the total weight of the SBV, Mouktik Bhasma in an amount ranging from 20 wt. % to 35 wt. % of the total weight of the SBV, Guduchi sattva in an amount ranging from 45 wt. % to 60 wt. % of the total weight of the SBV, and at least one excipient in an amount ranging from 5 wt. % to 30 wt. % of the total weight of the SBV.

Suvarna Bhasma is known as incinerated gold and Mouktik Bhasma is known as incinerated pearl. Suvarna Bhasma is sub-therapeutic in amount below 2 wt. %, and in amounts greater than 7 wt. %, there is an overload of the Bhasma which will be excreted. Similarly, the lower and upper weight percentages of the other ingredients i.e. Mouktik Bhasma and Guduchi Sattva are triturated in this composition keeping the above principle in mind.

The ingredients in powder form are blended together. The natural gum is added to the powder blend to form a dough along with purified water. Pellets having average weight of 5 gm are formed from this dough. The pellets are tray-dried typically at temperature in the range of 40° C. to 45° C. The dried pellets are granulated in a mixer grinder and the dry granules are taken for compression tableting. The average weight of the uncoated tablets is 240 mg±5%. The typical shelf life of the tablets is 3 years.

Guduchi is the common name for Tinospora. The Tinospora (Guduchi) plant is selected from Tinospora cordifolia and Tinospora sinensis. Particularly the stem of the plant is used for making the Sattva.

Tinospora cordifolia is also known as Guduchi of the family Menispermaceae. Tinospora cordifolia is indigenous to the tropical areas of India, Myanmar, and Sri Lanka. Tinospora cordifolia is obtained from Bharatiya Sanskriti Darshan Trust (BSDT), Wagholi, Pune.

Tinospora sinensis is also known as Malabar Gulbel or Gulvel of the family Menispermaceae. Tinospora sinensis is found in India, China, Sri Lanka, Nepal, Cambodia, Thailand, Vietnam, and Myanmar. Tinospora sinensis is obtained from Bharatiya Sanskriti Darshan Trust (BSDT), Wagholi, Pune.

In an embodiment of the present disclosure, the excipient is binder. The binder is selected from the group consisting of gum acacia, guar gum, and xanthan gum.

The SBV is administered orally in a dose of 250 mg/day to 450 mg/day.

In an exemplary embodiment of the present disclosure SBV is prepared from Suvarna Bhasma in an amount of 4.22 wt. % of the total weight of the SBV, Mouktik Bhasma in an amount of 26.37 wt. % of the total weight of the SBV, Guduchi Sattva in an amount of 52.74 wt. % of the total weight of the SBV and gum acacia in an amount of 16.67 wt. % of the total weight of the SBV.

The MKV in the present pharmaceutical composition comprises Mouktik Bhasma in an amount ranging from 10 wt. % to 14 wt. % of the total weight of the MKV, Shankha Bhasma in an amount ranging from 10 wt. % to 14 wt. % of the total weight of the MKV, Shouktik Bhasma in an amount ranging from 10 wt. % to 14 wt. % of the total weight of the MKV, Kapardik Bhasma in an amount ranging from 10 wt. % to 14 wt. % of the total weight of the MKV, Praval Bhasma in an amount ranging from 10 wt. % to 14 wt. % of the total weight of the MKV, Guduchi sattva in an amount ranging from 10 wt. % to 14 wt. % of the total weight of the MKV, Shudhha Gairik in an amount ranging from 10 wt. % to 14 wt. % of the total weight of the MKV, and at least one excipient in an amount ranging from 10 wt. % to 25 wt. % of the total weight of the MKV.

Mouktik Bhasma is known as incinerated pearl. The quantities below 10 wt. % the Mouktik Bhasma are sub-therapeutic and in quantities greater than 14 wt. %, there is an overload of the Bhasma which will be excreted. Similarly, the lower and upper weight percentages of the other ingredients i.e. Praval Bhasma, Shankha Bhasma, Shouktik Bhasma, Kapardik Bhasma, Shudhha Gairik, and Guduchi Sattva are triturated in this composition keeping the above principle in mind.

The ingredients in powder form are blended together. The natural gum is added to the powder blend to form a dough using purified water. Pellets having average weight of 5 gm each are formed from this dough. The pellets are tray-dried typically at temperature in the range of 40° C. to 45° C. The dried pellets are granulated in a mixer grinder and the dry granules are taken for compression tableting. The average weight of each uncoated tablet is 300 mg±5%. The typical shelf life of the tablets is 3 years.

The Tinospora (Guduchi) plant is selected from Tinospora cordifolia and Tinospora sinensis.

Tinospora cordifolia is also known as Guduchi of the family Menispermaceae. Tinospora cordifolia is indigenous to the tropical areas of India, Myanmar, and Sri Lanka. It is obtained from Bharatiya Sanskriti Darshan Trust, Wagholi, Pune.

Tinospora sinensis is also known as Malabar Gulbel or Gulvel of the family Menispermaceae. Tinospora sinensis is found in India, China, Sri Lanka, Nepal, Cambodia, Thailand, Vietnam, and Myanmar. It is obtained from Bharatiya Sanskriti Darshan Trust, Wagholi, Pune.

In an embodiment, the MKV is prepared from equal quantities of Mouktik Bhasma, Shankha Bhasma, Shouktik Bhasma, Kapardik Bhasma, Praval Bhasma, Guduchi Sattva and Shudhha Gairik and at least one edible binder, typically a natural gum, in an amount ranging from 10 wt. % to 30 wt. % of the total weight of the MKV.

In an embodiment of the present disclosure, the excipient is a binder, selected from the group consisting of gum acacia, guar gum, and xanthan gum.

The MKV is administered orally in a dose of 300 mg/day to 600 mg/day.

In an exemplary embodiment of the present disclosure MKV is prepared from equal quantities of Mouktik Bhasma, Shankha Bhasma, Shouktik Bhasma, Kapardik Bhasma, Praval Bhasma, Guduchi Sattva and Shudhha Gairik i.e. 12 wt. % each, and at least one edible binder, typically a natural gum, in an amount of 16 wt. % of the total weight of the MKV.

The Shatavari Vati (SV) prepared in accordance with the present disclosure comprises powder obtained from dried roots of Shatavari (Asparagus racemosus) in an amount ranging from 75 wt. % to 92 wt. % of the total weight of the SV, wherein the powder has a particle size in the range of 150 to 180 microns, and at least one excipient in an amount ranging from 8 wt. % to 25 wt. % of the total weight of the SV.

Shatavari powder is mixed with natural gum to form a dough along with purified water. Pellets having average of 5 gm are formed from this dough. These pellets are tray dried typically at temperature in the range of 40 to 45° C. The dried pellets are granulated in a mixer grinder and the dry granules are taken for compression tableting. The average weight of the uncoated tablets of Shatavari Vati is 300 mg±5%. The typical shelf life of these tablets is 3 years.

Shatavari is Asparagus racemosus. Asparagus racemosus is commonly used as Shatavari. Asparagus racemosus is of the family Liliaceae. It is found in throughout India in hilly areas as well as cultivated as a commercial commodity. Asparagus racemosus is obtained from Bharatiya Sanskriti Darshan Trust (BSDT), Wagholi, Pune.

In an embodiment of the present disclosure, the excipient is gum acacia.

The Shatavari Vati is administered orally in a dose of 300 mg/day to 600 mg/day.

The Arogya Vardhini Vati (AVV) prepared in accordance with the present disclosure comprises Kajjali in an amount ranging from 2.3 wt. % to 2.5 wt. % of the total weight of the AVV; loha Bhasma, Abhrak Bhasma, Tamra Bhasma, each in an amount ranging from 1.1 wt. % to 1.3 wt. % of the total weight of the AVV; dried fruit pericarp Haritaki, Bibhitaki, Amalaki powders, each in an amount ranging from 2.3 wt. % to 2.5 wt. % of the total weight of the AVV, Shuddha Shilajit powder in an amount ranging from 3.5 wt. % to 3.7 wt. % of the total weight of the AVV, Shuddha Guggul and Chitrak root powders, each in an amount ranging from 4.7 wt. % to 4.9 wt. % of the total weight of the AVV, Kutaki dried rhizome powder in an amount ranging from 26.2 wt. % to 26.8 wt. % of the total weight of the AVV, the extract from Nimba fresh leaf in an amount ranging from 22 wt. % to 22.4 wt. % of the total weight of the AVV, and at least one excipient in an amount ranging from 23.4 wt. % to 26.4 wt. % of the total weight of the AVV.

In an embodiment of the present disclosure, trituration is performed with Nimba fresh leaf juice in an amount ranging from 22 L to 24 L, wherein Kajjali, Tamra Bhasma, Abhrak Bhasma, loha Bhasma powders, each having a particle size in the range of 53 to 73 microns; Shuddha Guggul and Shuddha Shilajit powders, each having a particle size in the range of 355 to 450 microns; Haritaki, Bibhitaki, Amalaki, Chitrak, Kutaki powders, each having a particle size in the range of 150 to 180 microns and gum acacia powder having a particle size in the range of 150 to 180 microns.

In AVV, the ingredients, Kajjali, Tamra Bhasma, Abhrak Bhasma, loha Bhasma, Haritaki, Bibhitaki, Amalaki, Shuddha Shilajit, Shuddha Guggul, Chitrak and Kutaki are mixed properly and triturated by the fresh juice of Nimba leaf for 2 times. It is dried and then powdered. The final mixture obtained is mixed with specified amount of gum acacia powder and sufficient quantity of potable water are added to make a dough. Small pellets are made out of this dough and dried in oven at 45° C. The dried pellets are granulated and compressed into tablets of AVV, having weight of 335 mg with ±5% acceptable variation. The typical shelf life of these tablets is 5 years.

Haritaki is Terminalia chebula, Bibhitaki is Terminalia bellerica, Amalaki is Phyllathus emblica, Shuddha Guggul is purified Commiphora mukul, Chitrak is Plumbago zeylanica, Kutaki is Picrorrhiza kurroa and Nimba is Azadirachta indica. Other related species of each of the above plants also can be used in the herbo-mineral metallic pharmaceutical kit of the present disclosure.

Terminalia chebula (Gaertn.) Retz. is known as Haritaki belong to family Combretaceae. Plant is found throughout India chiefly in deciduous forests. It occurs abundantly in North India. Its range extends southwards at 300 to 900 m altitude.

Terminalia bellerica (Gaertn.) Roxb. (syn. T. punetata Roxb., Myrobalamus belerica B. Gaertn.) is known as Bibhitaki belong to family Combretaceae. The plant is common throughout India in plains and lower hills, chiefly in deciduous forests, at 900 m elevation where the climate is not very dry. It is also found in forests of Burma and Sri Lanka.

Phyllanthus emblica is known as Amalaki belong to family Phyllanthaceae/euphorbiaceae. Plant is deciduous tree found throughout India chiefly in deciduous forests or is widely cultivated.

Commiphora mukul/wightii, with common names Indian bdellium tree/mukul myrrh tree/gugal, is a flowering plant in the family Burseraceae, which produces a fragrant resin called Guggul. It is widely cultivated as a commercial commodity.

Plumbago zeylanica Linn. is also known as Chitraka belong to family Plumbaginaceae. It is a large perennial undershrub, found throughout India in plains and occasionally grown in the gardens.

Picrorhiza kurroa Royle ex Benth. is known as Katuka belong to family Scrophulariaceae. A small hairy herb with perenniating rhizomes growing in the hilly regions of the Northwest Himalayas in India, and Nepal at an altitude of 2700 to 4000 m.

Azadirachta indica/Melia azadirachta L. commonly known as neem, nimtree or Indian liliac, is a tree in the mahogany family Meliaceae. It is one of the two species in the genus Azadirachta and is native to the Indian subcontinent, and typically grown in tropical and semi-tropical regions.

Acacia nilotica (Linn.) Willd. Ex Delile ssp. Indica (Benth.) Brenan (syn. A. Arabica auct. Non Willd.) is known as Babbulah belong to family Mimosaceae. It is distributed throughout the warmer and dried parts of India. Drug consists of dried gummy exudate from the stem and branches.

In an embodiment of the present disclosure, the excipient is gum acacia.

The AVV is administered orally in a dose of 300 mg/day to 600 mg/day.

In an exemplary embodiment of the present disclosure, AVV is prepared from Kajjali in an amount of 2.4 wt. % of the total weight of the AVV; loha Bhasma, Abhrak Bhasma, Tamra Bhasma, each in an amount of 1.2 wt. % of the total weight of the AVV; Haritaki powder, Bibhitaki powder and Amalaki powder in an amount of 2.4 wt. % of the total weight of the AVV, Shuddha Shilajit in an amount 3.6 wt. % of the total weight of the AVV, Shuddha Guggul and Chitrak Powder in an amount of 4.8 wt. % of the total weight of the AVV, Kutaki powder in an amount of 26.4 wt. % of the total weight of the AVV, the dry extract from Nimba leaf juice in an amount of 22.2 wt. % of the total weight of the AVV, and gum acacia in an amount of 25 wt. % of the total weight of the AVV.

The Triphala Guggul Vati (TGV) comprises Haritaki dried pericarp, Bibhitaki dried pericarp, Amalaki dried pericarp and Pippali dried inflorescence powders, each in an amount ranging from 9.6 wt. % to 11.6 wt. % of the total weight of the TGV, dried purified Guggul exudate in an amount ranging from 50 wt. % to 56 wt. % of the total weight of the TGV, and at least one gliding agent, typically talc, in an amount ranging from 3.6 wt. % to 5.6 wt. % of the total weight of the TGV, wherein Haritaki, Bibhitaki, Amalaki and Pippali powders, each has a particle size in the range of 150 to 180 microns; Shuddha Guggul has a particle size in the range of 250-355 microns and talc powder has a particle size in the range of 75 to 105 microns.

In TGV, powders of Haritaki, Bibhitaki, Amalaki and Pippali are mixed with the slurry of Shuddha Guggul made in 2 times purified water (w/v) to obtain a dough. The dough is further pelletized and dried in oven. The dried pellets are grinded to obtain granules. Talc up to 5% is added to these granules and compressed in tablet punching machine to obtain compressed tablets of TGV weighing 262.5 mg each with ±5% acceptable variation. The typical shelf life of these tablets is 5 years.

Haritaki is Terminalia chebula, Bibhitaki is Terminalia bellerica, Amalaki is Phyllanthus emblica, Pippali is Piper longum, and Guggul Commiphora mukul. Other related species of each of the above plants also can be used in the herbo-mineral metallic pharmaceutical kit of the present disclosure.

Terminalia chebula (Gaertn.) Retz. is know as Haritaki belong to family Combretaceae. Plant is found throughout India chiefly in deciduous forests. It occurs abundantly in North India. Its range extends southwards at 300 to 900 m altitude.

Terminalia bellirica (Gaertn.) Roxb. (syn. T. punetata Roxb., Myrobalamus belerica B. Gaertn.) is known as Bibhitaki belong to family Combretaceae. The plant is common throughout India in plains and lower hills, chiefly in deciduous forests, at 900 m elevation where the climate is not very dry. It is also found in forests of Burma and Sri Lanka.

Phyllanthus emblica is known as Amalaki belong to family Phyllanthaceael euphorbiaceae. Plant is a deciduous tree found throughout India chiefly in deciduous forests or is widely cultivated.

Piper longum Linn. is known as Pippali belong to family Piperaceae. The plant is a slender climber. Distributed in warmer regions of the country i.e. Western Ghats, central Himalayas to Assam, Khasi and Miker hills and lower hills of Bengal.

Commiphora mukul/wightii, with common names Indian bdellium tree/mukul myrrh tree/gugal, is a flowering plant in the family Burseraceae, which produces a fragrant resin called Guggul. It is widely cultivated as a commercial commodity.

In an embodiment of the present disclosure, the gliding agent is talcum powder.

The TGV is administered orally, in a dose of 300 mg/day to 600 mg/day.

In an exemplary embodiment of the present disclosure, TGV is prepared from Amalaki powder, Bibhitaki powder, Haritaki powder, each in an amount of 10.6 wt. % of the total weight of the TGV, Shuddha Guggul in an amount of 53 wt. % of the total weight of the TGV, and at least one gliding agent, typically talc, in an amount of 4.6 wt. % of the total weight of the TGV.

All the plants used in the polyherbal composition are obtained from Bharatiya Sanskriti Darshan Trust (BSDT), Wagholi, Pune.

The jatamansi Oil (JMO) prepared in accordance with the present disclosure comprises extract of jatamansi obtained from dried rhizomes of jatamansi (Nardostachys jatamansi) in an amount ranging from 0.5 wt. % to 2 wt. % of the total weight of the JMO, wherein the extract is derived from the decoction of jatamansi, and sesame oil is used as base in an amount ranging from 99.5 wt. % to 98 wt. % of the total weight of the JMO.

jatamansi coarse powder sieved through 2 mm mesh is soaked in 8 times water for 18 h and decoction is prepared by reducing it to one half. It is mixed with sesame oil in ratio of 1:1 (v/v) and heated at 100° C. till the water evaporates. The obtained product is strained and self-cooled to get JMO. The typical shelf life of the oil is 3 years.

jatamansi is jatamansi Nardostachys. Nardostachys jatamansi is commonly used as jatamansi or Indian spikenard. Nardostachys jatamansi is of the family Valerianaceae. It is found in Himalayan ranges in India as well as cultivated as a commercial commodity. Nardostachys jatamansi and sesame oil were obtained from Bharatiya Sanskriti Darshan Trust (BSDT), Wagholi, Pune.

In an embodiment of the present disclosure, the base is sesame oil. The jatamansi Oil is applied externally on the scalp in quantity as recommended.

The foregoing description of the embodiments has been provided for purposes of illustration and not intended to limit the scope of the present disclosure. Individual components of a particular embodiment are generally not limited to that particular embodiment, but, are interchangeable. Such variations are not to be regarded as a departure from the present disclosure, and all such modifications are considered to be within the scope of the present disclosure.

The present disclosure is further described in light of the following experiments which are set forth for illustration purpose only and not to be construed for limiting the scope of the disclosure. The following experiments can be scaled up to industrial/commercial scale and the results obtained can be extrapolated to industrial scale.

### EXPERIMENTAL DETAIL

### Example 1

The components of the herbo-mineral metallic pharmaceutical kit containing oral medicines are given in Table-1.

TABLE 1
Components of the herbo-
mineral metallic pharmaceutical kit

| Sr. | | Dosage | |
|---|---|---|---|
| No. | Components | administered | Frequency of dose |
| 1 | Suvarna | 395 mg | Morning after breakfast |
| | Bhasmadi Vati | (2 tablets) | (8.00AM)-Evening |
| | (SBV) | | after snacks (5.00PM) |
| 2 | Mouktikyukta | 500 mg | Morning after breakfast |
| | Kamdudha Vati | (2 tablets) | (8.00AM)-Evening |
| | (MKV) | | after snacks (5.00PM) |
| 3 | Shatavari | 500 mg | Morning after breakfast |
| | Vati (SV) | (2 tablets) | (8.00AM)-Evening |
| | | | after snacks (5.00PM) |
| 4 | Arogyavardhini | 500 mg | After Lunch |
| | Vati | (2 tablets) | and Dinner |
| | (AVV) | | |
| 5 | Triphala Guggul | 500 mg | After Lunch |
| | Vati (TGV) | (2 tablets) | and Dinner |
| 6 | Jatamansi Oil | 10 ml | For a special type of |
| | (JMO) | | head massage |
| | | | (Shiropichu) at bedtime |

Experiment 1.1: Composition of Suvarna Bhasmadi Vati (SBV) in Accordance with the Present Disclosure

SBV was prepared using the following ingredients as given in Table-2.

TABLE 2
Composition of Suvarna Bhasmadi Vati (SBV)

| | | | Quantity | Quantity | Quantity |
|---|---|---|---|---|---|
| | | | for 5 kg | for 5 kg | for 5 kg |
| Sr. | | Latin name/ | batch- | batch- | batch- |
| No. | Contents | English Name | Batch 1 | Batch 2 | Batch 3 |
| 1 | Suvarna Bhasma | Incinerated Gold | 211 g | 110 g | 350 g |
| 2 | Mouktik Bhasma | Incinerated Pearl | 1319 g | 1700 g | 1000 g |
| 3 | Aqueous extract | Starch of Tinospora | 2637 g | 2940 g | 2250 g |
| | of Tinospora | cordifolial sinensis/ | | | |
| | cordifolia/ | Tinospora/Heart | | | |
| | sinensis | leaved moonseed | | | |
| 4 | Gum Acacia | | 833 g | 250 g | 1400 g |
| | powder | | | | |

### Process for Preparing Suvarna Bhasmadi Vati (SBV):

### Step 1: Preparation of Suvarna Bhasma

Initially, 300 gm of Suvarna foils were amalgamated with 2500 gm of metallic mercury and 2500 gm of sulphur powder having particle size 150 microns and then incinerated 18 times each at 650° C. for 6 hours to get incinerated Suvarna. For stabilization, the incinerated Suvarna was triturated for 6 hours with 200 ml fresh juice of the leaves of Ocimum sanctum (tulsi/Holy basil) and further incinerated at 600° C. for 5 hours. This process of incineration in tulsi juice was repeated 25 times to obtain Suvarna Bhasma having particle size in the range of 20-500 nm (average particle size 350 nm). The particle size of the so obtained Suvarna Bhasma was analysed by particle size analyser (90 plus from Brookhaven Instruments, USA).

tulsi—Ocimum sanctum is also known as holy basil of the family Lamiaceae. Ocimum sanctum was obtained from Bharatiya Sanskriti Darshan Trust (BSDT), Wagholi, Pune.

### Specification of Suvarna Bhasma:

Description: Brown coloured, very fine free flowing powder
Loss on Drying—NMT 0.5% w/w
Loss on Ignition—NMT 1% w/w
Acid insoluble ash—90 to 98% w/w
Assay as Au—NLT 85% w/w

### Step 2: Preparation of Mouktik Bhasma

8 Kg Mouktik (pearl) was boiled in 32 L of butter milk having curd to water ratio of 1:2 w/v and pH 3, to obtain purified Mouktik. The so obtained purified Mouktik was powdered and triturated with 4 L of rose water to obtain triturated powder. The triturated powder was then incinerated using cow-dung cakes at 700° C. to obtain Mouktik Bhasma (incinerated pearl). Average particle size of Mouktik Bhasma was 55-73 μm.

### Specification of Mouktik Bhasma:

Description: Greyish white coloured, very fine powder
Loss on Drying—NMT 0.5% w/w
Acid insoluble ash—NMT 2% w/w
Calcium assay—38 to 40% w/w
pH—10 to 11
   Step 3: Extraction of Starch from Tinospora cordifolia

50 Kg of fresh stems of Tinospora cordifolia were chopped into small pieces. These pieces were crushed and then soaked for 12 hours in 4 times (w/v) of potable water (200 L) in a stainless steel vessel. The mixture was macerated in water thoroughly and filtered slowly to obtain solution containing aqueous extract of Tinospora cordifolia. The solution thus obtained was kept aside for 12 hours to obtain supernatant and smooth starchy sediment of Tinospora cordifolia. The supernatant was carefully separated to obtain smooth starchy sediment. The smooth starchy sediment of Tinospora cordifolia was evaporated in an oven at 45° C. to obtain Guduchi Sattva in the form of dry starch.

Specification of Starch Extract of Tinospora cordifolia:
Description: Greyish white coloured very fine free flowing starchy powder
Loss on Drying—NMT 5% w/w
Acid insoluble ash—NMT 1% w/w
Gelation temperature −60 to 75° C.

### Step 4: Preparation of SBV

In a mixer, Suvarna Bhasma, Mauktik Bhasma, Guduchi Sattva (starch from Tinospora cordifolia), and gum acacia were mixed in a proportion given in Table 2. To this mixture, sufficient amount of water was added to obtain a dough. The dough was further pelletized to obtain pellets. The pellets were dried in oven at 45° C. to obtain dried pellets. The dried pellets were grinded to obtain granules having powder to granule ratio of 30:70. The mixture of granules and powder was compressed in tablet punching machine to obtain compressed tablet of weight 237±5 mg.

### Specifications of SBV:

| | |
|---|---|
| Appearance | Grey colour, round |
| Shape | Biconvex tablets |
| Weight variation | 0.2210 to 0.2300 |
| Average weight | 0.2300 to 0.2500 |
| Hardness | 1-2 Kg/cm² |
| Friability | NMT 1% w/w |
| Disintegration Time | NMT 30 min |
| Diameter | 7 to 7.5 mm |
| Width | 3.3 to 4.6 mm |
| Acute toxicity | LD 50 > 2000 mg/kg |

Experiment 1.2: Composition of Kamdudha Mouktikyukta Vati (MKV) in Accordance with the Present Disclosure

MKV was prepared using the following ingredients as given in Table-3.

TABLE 3
Composition of Kamdudha Mouktikyukta Vati (MKV)

| | | | Quantity | Quantity | Quantity |
|---|---|---|---|---|---|
| | | | for 5 kg | for 5 kg | for 5 kg |
| Sr. | | Latin name/ | batch- | batch- | batch- |
| No. | Contents | English Name | Batch 1 | Batch 2 | Batch 3 |
| 1 | Mouktik Bhasma | Incinerated Pearl | 3572 g | 3500 | 4000 |
| 2 | Shankha Bhasma | Incinerated Conch | 3572 g | 3000 | 3800 |
| 3 | Shouktik Bhasma | Incinerated | 3572 g | 3000 | 3800 |
| | | Pearl Shell | | | |
| 4 | Kapardik Bhasma | Incinerated Cowrie | 3572 g | 3000 | 3800 |
| 5 | Praval Bhasma | Incinerated Coral | 3572 g | 3500 | 3800 |
| 6 | An aqueous | Starch of Tinospora | 3572 g | 3500 | 4000 |
| | extract of | cordifolial sinensis | | | |
| | Tinospora | | | | |
| | cordifolia/ | | | | |
| | sinensis | | | | |
| 7 | Shudhha Gairik | Red ochre roasted in | 3572 g | 3000 | 3800 |
| | | cow ghee | | | |
| 8 | Gum Acacia | | 5000 g | 7500 | 3000 |
| | powder | | | | |

### Process for Preparing Kamdudha Mouktikyukta Vati (MKV):

All the five Mouktik Bhasma, Shankha Bhasma, Shouktik Bhasma, Kapardik Bhasma, Praval Bhasma are prepared using methods known to person skilled in the art. The five bhasmas, starch of Tinospora cordifolia/sinensis, Shudhha Gairik and gum acacia powder were mixed in a proportion given in Table 3. To this mixture, sufficient amount of potable water was added to obtain a dough. The dough was further pelletized to obtain pellets. The pellets were dried in oven at 45° C. to obtain dried pellets. The dried pellets were grinded to obtain granules having powder to granule ratio of 30:70. The mixture of granules and powder was compressed in tablet punching machine to obtain compressed tablet of weight 300±5 mg.

### Specification of Shankha Bhasma:

Description—Greyish white very fine powder
Loss on drying—NMT 1% w/w
Acid insoluble ash—NMT 2% w/w
pH—9 to 10
Calcium assay as Ca—38 to 40% w/w

### Specification of Shouktik Bhasma:

Description—Greyish white very fine powder
Loss on drying—NMT 1% w/w
Acid insoluble ash—NMT 2% w/w
pH—10 to 11
Calcium assay as Ca—38 to 40% w/w

### Specification of Kapardik Bhasma:

Description—Greyish white very fine powder
Loss on drying—NMT 1% w/w
Acid insoluble ash—NMT 2% w/w
pH—10 to 11
Calcium assay as Ca—38 to 40% w/w

### Specification of Praval Bhasma:

Description—Greyish white very fine powder
Loss on drying—NMT 1% w/w
Acid insoluble ash—NMT 2% w/w
pH—10 to 11
Calcium assay as Ca—40 to 45% w/w

### Specification of Mouktik (Pearl) Bhasma:

Description—Greyish white very fine powder
Loss on drying—NMT 1% w/w
Acid insoluble ash—NMT 2% w/w
pH—10 to 11
Calcium assay as Ca—38 to 40% w/w

### Specification of Shudhha Gairik (Red Ochre):

Loss on drying—NMT 1% w/w
Iron assay—NLT 15% w/w
Silica assay—18 to 20% w/w
Oil content—3 to 3.5% w/w
   Specification of Guduchi Sattva (Starch of Tinospora cordifolia/sinensis):
Loss on drying—4 to 5% w/w
Acid Insoluble ash—NMT 1% w/w
Gelation temperature—60 to 75° C.

### Specification of Kamdudha Mouktikyukta Vati (MKV):

| | |
|---|---|
| Description | Light brown colour |
| Shape | Round biconvex tablet |
| Weight variation | 0.2850 to 0.3150 |
| Average weight | 0.2900 to 0.3100 |
| Hardness | 2 to 4 Kg/cm² |
| Friability | NMT 1% w/w |
| Disintegration Time | NMT 30 min |
| Diameter | 7 to 8 mm |
| Width | 3 to 4 mm |
| Acute Toxicity | LD 50 > 2000 mg/kg |

Experiment 1.3: Composition of Shatavari Vati (SV) in Accordance with the Present Disclosure

Shatavari Vati was prepared using the following ingredients as given in Table-4.

TABLE 4
Composition of Shatavari Vati

| Sr. | | Common | Quantity for | Quantity for | Quantity for |
|---|---|---|---|---|---|
| No. | Contents | Name | 30 kg batch | 30 kg batch | 30 kg batch |
| 1 | Powder of | Shatavari | 25000 g | 23000 g | 27000 g |
| | Asparagus | | | | |
| | racemosus | | | | |
| 2 | Gum Acacia | | 5000 g | 7000 g | 3000 g |
| | powder | | | | |

### Process for Preparing Shatavari Vati (SV):

In a mass mixer, powders of Asparagus racemosus and gum acacia were mixed in a proportion given in Table 4. To this mixture, sufficient amount of water was added to obtain a dough. The dough was further pelletized to obtain pellets. The so obtained pellets were dried in oven at 45° C. to obtain dried pellets. The dried pellets were grinded to obtain granules having powder to granule ratio of 30:70. The so obtained granules and powder were compressed in tablet punching machine to obtain compressed tablet of weight 300 mg±5% variation.

### Specification of Shatavari Vati (SV):

| | |
|---|---|
| Description | Buff coloured uncoated, round, |
| | biconvex tablet having characteristic odour. |
| Weight variation | 0.290-0.310 g |
| Average weight | 0.300 g |
| Hardness | 2-2.5 Kg/cm² |
| Friability | NMT 1% w/w |
| Disintegration Time | NMT 15 min |
| Diameter | 9.5-10.5 mm |
| Width | 4-5 mm |
| Loss on drying | Not more than 8% w/w |
| Total Ash | 3-7% w/w |
| Acid Insoluble Ash | 0.5-3% w/w |
| Water Soluble extractive | 40-50% w/w |
| Alcohol soluble extractive | 12-16% w/w |
| Microbial limits | Total Viable Count (TVC)—NMT 10⁵/gm |
| | Yeast and Moulds—10³/gm |
| | Fungal count—Absent |
| Heavy metal limits | Not more than 20 ppm |
| Thin Layer Chromatography | n-Butenol:Acetic acid:Water:: 4:1:5 |
| | I2 vapours—2 spots—Rf: 0.44, 0.56 (Yellow) |
| | 5% methanolic sulphuric acid—5 spots— |
| | 0.1, 0.34 (Black), 0.45, 0.56 (Yellow), |
| | 0.87(Brown) |

Experiment 1.4: Composition of Arogyavardhini Vati (AVV) in Accordance with the Present Disclosure

AVV was prepared using the following ingredients as given in Table-5.

TABLE 5
Ingredients used for preparing Arogyavardhini Vati (AVV)

| | | | | Qty. of | Qty. of | Qty. of |
|---|---|---|---|---|---|---|
| | | | | raw | raw | raw |
| | | | | materials | materials | materials |
| | | | Form used | used in | used in | used in |
| S. | | | (Particle | 15.625 Kg- | 15.625 kg- | 15.625 kg- |
| No. | Ingredient | Part used | size- μm) | Batch 1 | Batch 2 | Batch 3 |
| 1 | Kajjali | Triturated | Powder | 400 g | 400 g | 400 g |
| | | Powder | (53-75) | | | |
| 2 | Tamra Bhasma | Incinerated | | 200 g | 150 g | 250 g |
| 3 | Abhrak Bhasma | | | 200 g | 150 g | 250 g |
| 4 | Loha Bhasma | | | 200 g | 150 g | 250 g |
| 5 | Haritaki | Fruit pericarp | Powder | 400 g | 350 g | 450 g |
| 6 | Bibhitaki | Fruit pericarp | (180-250) | 400 g | 350 g | 450 g |
| 7 | Amalaki | Fruit pericarp | | 400 g | 350 g | 450 g |
| 8 | Shuddha Shilajit | Processed | Powder | 600 g | 550 g | 650 g |
| | | exudate | (355-425 μm) | | | |
| 9 | Shuddha Guggul | Processed | | 800 g | 750 g | 850 g |
| | | exudate | | | | |
| 10 | Chitrak | Root | Powder | 800 g | 750 g | 850 g |
| 11 | Kutaki | Rhizome | (180-250) | 4400 g | 4300 g | 4500 g |
| 12 | Nimba | Leaf | Dry extract | 3700 g | 3300 g | 4100 g |
| | (Trituration | | (Fresh | (24 L) | (21.5 L) | (26.5 L) |
| | media) | | juice) | | | |
| 13 | Gum acacia | Exudate | Powder | 3125 g | 4075 g | 2175 g |
| | (Excipient) | | (150-180) | | | |

### Process for Preparing Arogyavardhini Vati (AVV):

### Step I: Trituration of Herbs, Metals and Mineral Drugs

The ingredients 1 to 11 (Table 5) were mixed properly in a stone mortar with pestle for 3 hr. This mixture was triturated twice by the fresh juice of ingredients 12 (Table 5) for 6 hrs each time and dried after each trituration in oven at 45° C. and then powdered fine to pass through mesh no. 40.

### Step II: Tableting of Final Product Obtained in Step I

The final mixture obtained in Step I was mixed with specific amount of gum acacia powder per batch (as per Table 5) and sufficient quantity of potable water was added to make a dough. Small pellets were made out of this dough and dried in oven at 45° C. The dried pellets were granulated to form granules having granule to powder ratio of 70:30. The obtained granules were compressed into tablets of AVV, having weight of 335 mg with ±5% acceptable variation.

### Specification of Arogyavardhini Vati (AVV):

| | |
|---|---|
| Description | Dark brown colour, round, biconvex |
| | tablets having characteristic odour |
| | with logo mark on one side |
| Weight variation | 0.3170-0.3340 |
| Average weight | 0.3225-0.3400 |
| Hardness | 2-4 Kg/cm² |
| Friability | NMT 1% w/w |
| Disintegration Time | NMT 45 min |
| Diameter | 7-7.5 mm |
| Width | 5-5.5 mm |
| Total Ash | 9-11% w/w |
| Acid Insoluble Ash | 3.5-4% w/w |
| Water Soluble extractive | 30-40% w/w |
| Alcohol soluble extractive | 2-4% w/w |
| Microbial limits | Total Viable Count (TVC)—NMT 10⁵/gm |
| | Yeast and Moulds—NMT 10³/gm |
| | E. coli—Absent |
| Elemental Assay | Hg—1-1.5% w/w |
| | Cu—0.5-1% w/w |
| | Fe—0.75-1.25% w/w |
| | S—1.75-2.25% w/w |

Experiment 1.5: Composition of Triphala Guggul Vati (TGV) in Accordance with the Present Disclosure

TGV was prepared using the following ingredients as given in Table-6.

TABLE 6
Ingredients used for preparing TGV

| | | | | Qty. of | Qty. of | Qty. of |
|---|---|---|---|---|---|---|
| | | | | raw | raw | raw |
| | | | | materials | materials | materials |
| | | | Form used | used in | used in | used in |
| S. | | | (Particle | 3.780 Kg- | 3.780 kg- | 3.780 kg- |
| No. | Ingredient | Part used | size- μm) | Batch 1 | Batch 2 | Batch 3 |
| 1 | Haritaki | Fruit pericarp | Powder | 400 g | 350 g | 450 g |
| 2 | Bibhitaki | Fruit pericarp | (180-250) | 400 g | 350 g | 450 g |
| 3 | Amalaki | Fruit pericarp | | 400 g | 350 g | 450 g |
| 4 | Pippali | Inflorescence | | 400 g | 350 g | 450 g |
| 5 | Shuddha Guggul | Processed | Powder | 2000 g | 2300 g | 1700 g |
| | | exudate | (355-450) | | | |
| 6 | Talc (Gliding | Exudate | Powder | 180 g | 80 g | 280 g |
| | agent) | | (150-180) | | | |

### Process for Preparing Triphala Guggul Vati (TGV):

Ingredient 5 was soaked in twice quantity of boiling water (w/v) overnight to get thick slurry. Powders of all the ingredients from 1 to 4 were mixed in a proportion given in Table 6 into the slurry of ingredient 5 so as to obtain a dough. The dough was further pelletized to obtain pellets. The pellets were dried in oven at 45° C. to obtain dried pellets. There was no appreciable weight gain. The dried pellets were grinded to obtain granules having granule to powder ratio of 70:30. The granules and powder were compressed in tablet punching machine to obtain compressed tablets of TGV weighing 262.5 mg each with ±5% acceptable variation.

### Specification of Triphala Guggul Vati (TGV):

| | |
|---|---|
| Description | Grey colour biconvex, cylindrical tablets |
| Average weight | 255-270 mg |
| Diameter | 8 mm |
| Height | 4 mm |
| Friability | Not more than 1% w/w |
| Disintegration time | Not more than 45 min |
| Loss on Drying | Not More Than 10% |
| Total Ash | Not More Than 20% |
| Acid Insoluble Ash | Not More Than 8% |
| Water Soluble extractive | Not Less Than 30% w/w |
| Alcohol soluble extractive | Not Less Than 8% w/w |
| Microbial limits | Total Viable Count (TVC)—NMT 10⁵/gm |
| | Yeast and Moulds—NMT 10³/gm |
| | E. coli—Absent |
| Heavy metal limits | Not more than 20 ppm |
| Thin Layer Chromatography | Toluene:Acetone:: 9:1 |
| | UV 365—07 spots Rf: 0.045 (Red), 0.090 |
| | (Blue), 0.11, 0.18, 0.29 (All Flu Blue), |
| | 0.42 (Blue), 0.50 (Flu Blue) |
| | Anisaldehyde Sulphuric acid reagent 08 |
| | spots Rf: 0.092 (Green), 0.14 (Blue), |
| | 0.24 (Pink), 0.37 (Violet), 0.43 (Brown), |
| | 0.54 (Violet), 0.81, 0.99 (Blue) |

Experiment 1.6: Composition of jatamansi Oil (JMO) in Accordance with the Present Disclosure

JMO was prepared using the following ingredients as given in Table-7.

TABLE 7
Ingredients used for preparing JMO

| | | | | Qty. of | Qty. of | Qty. of |
|---|---|---|---|---|---|---|
| | | | | raw | raw | raw |
| | | | | materials | materials | materials |
| | | | Form used | used in | used in | used in |
| S. | | | (Particle | 40 Lit.- | 3.780 kg- | 3.780 kg- |
| No. | Ingredient | Part used | size- μm) | Batch 1 | Batch 2 | Batch 3 |
| 1 | Jatamansi | Dried rhizome | Coarse | 10 Kg | 5 Kg | 15 Kg |
| | | | powder | | | |
| | | | (2 mm) | | | |
| 2 | Til Taila | Seed oil | — | 40 Lit | 40 Lit | 40 Lit |

Process for Preparing jatamansi Oil (JMO):

Coarse powder of dried rhizomes of jatamansi sieved through 2 mm mesh was soaked in water, wherein the ratio of jatamansi to water is 1:8 w/v, for 18 h. Decoction is prepared by reducing it to one half. The decoction was mixed with sesame oil in ratio of 1:1 (v/v) and boiled at 100° C. till the water evaporated. The obtained product was strained and self-cooled to get JMO.

Specification of jatamansi Oil (JMO):

| | |
|---|---|
| Description | Slightly yellowish colored thick viscous |
| | liquid (oil) |
| Specific gravity at 25° C. | 0.9100-0.9300 |
| Refractive index at 25° C. | 1.5400-1.500 |
| Acid value | 2.825-3.200 |
| Saponification value | 100-170 |
| Iodine value | 60-150 |
| Peroxide value | 0.5-2.5 |
| Microbial limits | Total Viable Count (TVC)—NMT 10⁵/gm |
| | Fungal count—10/gm |
| Heavy metal limits | Not more than 20 ppm |
| Thin Layer Chromatography | Toluene:Ethyl acetate:: 9:1 |
| | UV 254 nm—02 spots Rf: 0.32, 0.64 |
| | (Both blue) |
| | Iodine vapours—04 spots Rf: 0.27, 0.34, |
| | 0.40, 0.63 (All yellow) |
| | Anisaldehyde sulphuric acid—04 spots |
| | Rf: 0.27, 0.34, 0.40, 0.89 (All light blue) |

### Example 2

### Efficacy Study of the Herbo-Mineral Metallic Pharmaceutical Kit of the Present Disclosure:

In this study, 27 Triple Negative Breast Cancer patients were included. These patients were treated with the composition of herbo-mineral metallic pharmaceutical kit after completion of their conventional treatment.

Inclusion Criteria for Enrolling Patients in this Study

Female patients: Triple Negative Breast Cancer patients of all stages in age group between 18 to 65 years after completing their conventional treatment.

Exclusion Criteria for Enrolling Patients in this Study

Patients who were on other Ayurvedic drugs for breast cancer or any other ailment and patients who had distant metastasis and/or recurrence.

TABLE 8
Demographic data of patients enrolled in this study

| | | No. of patients |
|---|---|---|
| Parameters | | (n = 27) |
| Age group (In yrs.) at | 25-30 | 0 |
| diagnosis between- | 31-40 | 10 |
| | 41-50 | 9 |
| | 51-60 | 7 |
| | 61-65 | 1 |
| | Total | 27 |
| Grade | I | 1 |
| | II | 12 |
| | III | 14 |
| | Total | 27 |
| Stage | IA | 2 |
| | IB | 0 |
| | IIA | 11 |
| | IIB | 7 |
| | IIIA | 4 |
| | IIIB | 1 |
| | IIIC | 2 |
| | Total | 27 |
| HP Type | IDC | 24 |
| | Clear cell ca | 1 |
| | Invasive lobular | 1 |
| | ca | |
| | Invasive | 1 |
| | mammary ca | |
| | Adenocarcinoma | 0 |
| | Metaplastic ca | 0 |
| | Total | 27 |
| Breast Conservative surgery/ | BCS | 13 |
| MRM of affected breast | MRM | 14 |
| | Not Done | 0 |
| | Total | 27 |
| Chemotherapy | Taken | 27 |
| | Not Taken | 0 |
| | Total | 27 |
| Radiotherapy | Taken | 23 |
| | Not advised | 4 |
| | Total | 27 |
| Family History | Yes | 10 |
| | No | 17 |
| | Total | 27 |

### Outcome Measures—

### Time Points for Assessment of Outcome Measures—

A—Before starting treatment of herbo-mineral metallic pharmaceutical kit (n=27)
B—one year after starting treatment of herbo-mineral metallic pharmaceutical kit (n=27),
C—two years after starting treatment of herbo-mineral metallic pharmaceutical kit (n=26),
D—three years after starting treatment of herbo-mineral metallic pharmaceutical kit (n=20).

### A. Clinical Investigations

The Patients were Followed for:
Assessment of performance status using Karnofsky score (Grading for well-being on 0 to 100 scale, higher score denotes better performance) and Weight.
Assessment of Quality of Life (QoL) using Questionnaire QLQ C30 (designed for all types of cancers) and BR 23 (specially designed for breast cancer patients) of EORTC determined on the basis of patients' own perspective about her well-being.
QLQ C30 can be interpreted as—
1. Symptomatology (Symptom score)
2. Ability to perform routine activities (Functional score)
3. Overall well-being (Global score)

(Karnofsky score and scoring for QOL are internationally accepted means of scoring symptoms and quality of life for cancer patients used in various studies in clinical trials.)

To support the clinical observations, additional studies were conducted using clinical laboratory investigations and basic laboratory investigations as follows:

### B. Clinical Laboratory Investigations:

These investigations were conducted for assessing hemoglobin status along with toxicity for liver and kidney on the basis of corresponding enzyme levels. CA 15.3—a Breast cancer specific tumor marker was studied to assess tumor burden (in view of higher chances of developing breast cancers in Triple Negative Breast Cancer patients). CRP levels were assessed as an indicator of initiation of inflammatory response.

The tests performed were
Haemogram
LFT (Liver Function Test)
KFT (Kidney Function Test)
Tumor marker CA 15.3
CRP Test

### C. Basic Laboratory Investigations:

These tests were carried out to provide proof of concept by assessing status of immunomodulatory effect and oxidative stress management with a herbo-mineral metallic pharmaceutical kit in these patients. Cytokines are the key molecules controlling proliferation, differentiation, and functions of immune cells and inflammatory response Immunological investigations—pro inflammatory cytokines—IL-1β, IL-6, IL-8 and IL-10 assessed by ELISA, using commercial kits.

The oxidative stress is considered to be involved in the pathophysiology of cancers. The activity of erythrocyte antioxidant defense enzymes Superoxide Dismutase (SOD) and Catalase, and Glutathione were studied by colorimetric assays, using commercial kits.

For scores and basic and clinical laboratory investigations unpaired ‘t’ test was applied.

### D. Radiological Investigation—PET CT

PET CT is done every year till completion of 3 years with the treatment of herbo-mineral metallic kit to assess disease status of TNBC patients.

### Experiment 2.1: Efficacy Studies of the Herbo-Mineral Metallic Pharmaceutical Kit Using Hematological and Biochemical Parameters

The clinical laboratory parameters studied were haemogram, LFT, KFT, CRP and tumor marker CA 15.3. All these parameters were within normal range in all the patients at all-time points. Given below were the trends at different time points. The results are based on mean values at each time point.

### Results—

### Hematological Parameters—

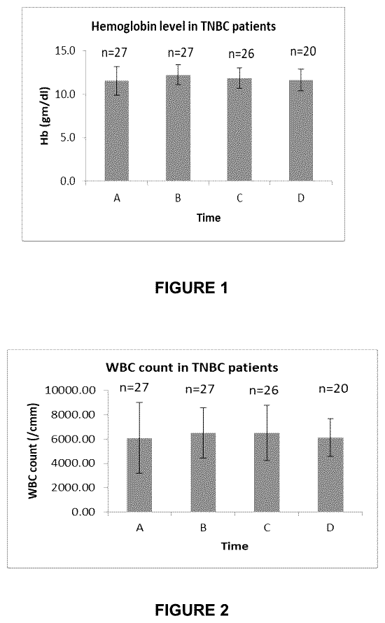

Hemoglobin—Average hemoglobin levels were in normal range throughout the treatment period (i.e. at timepoints B, C and D) (FIG. 1). Hemoglobin levels of three TNBC patients improved remarkably at the level of 12.3, 11.7 and 10.8 at the end of treatment who had baseline hemoglobin levels below normal range, i.e. 8, 8.9 and 7, respectively. Three TNBC patients had hemoglobin levels below normal range at time points C and D {9.2 (at time point C), 9.6 and 8.4 (at time point D)} due to chemotherapy induced myelo suppression. These three patients were treated with chemotherapy during these time points due to disease progression. One TNBC patient was diagnosed with Pre Acute Lymphoblastic Leukemia (Pre ALL) at time point C and thus had decreased level of hemoglobin (9.9).

WBC—Average WBC counts were in normal range throughout the treatment period (i.e. at time points B, C and D) (FIG. 2).

7 TNBC patients had low WBC counts due to chemotherapy induced myelo suppression (4 at time point A, one each at time points B, C and D). All of them had normal WBC counts at the remaining time points. one patient had higher WBC count at time point B due to Urinary Tract Infection (UTI), whereas another patient developed second malignancy (Pre ALL) at time point C and had low WBC count.

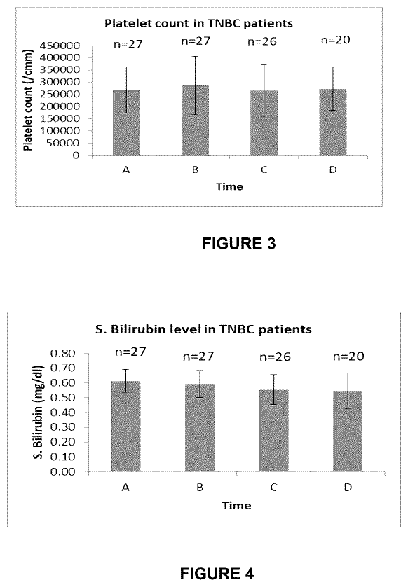

Platelets—Average platelet counts were in the normal range throughout the treatment (FIG. 3). However, in three TNBC patients it was below normal range due to chemotherapy induced myelo suppression (one at time point A and two at time points C). Chemotherapy was started in these two patients due to metastasis at time point C. 4^{th} patient also had low Platelet count at time point C due to development of second malignancy i.e. Pre-ALL.

### Biochemical Parameters—

### Liver Function Tests (LFT)

Serum Bilirubin (Total) and Serum alkaline Phosphatase—

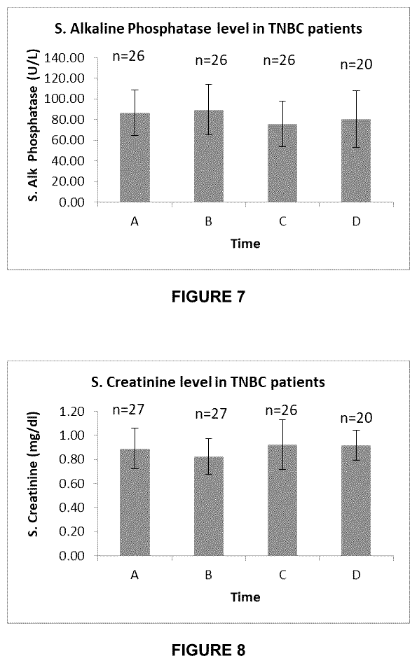

These levels remained within the normal range for all the patients for all the time points. (FIGS. 4 and 7).

### SGOT and SGPT—

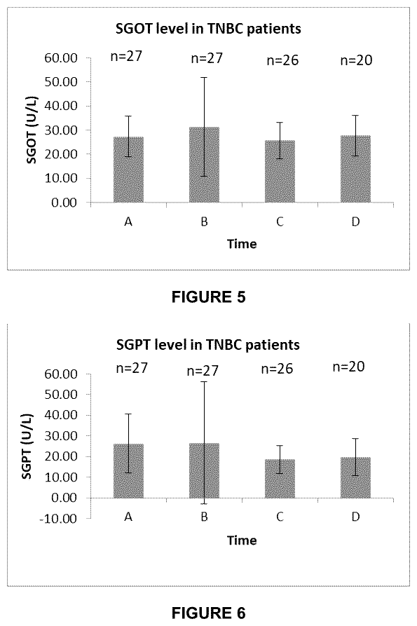

Average SGOT and SGPT levels remained within the normal range for all the patients at all the four time points with few exceptions (FIGS. 5 and 6). One patient had elevated level of SGOT and

SGPT at time point A and SGPT at time point B. This patient developed skeletal, lung and regional nodal metastasis at time point B. It is notable that SGOT and SGPT levels of this patient became within normal limits at time point C, in-spite of progression of disease. Another TNBC patient also had raised SGOT and SGPT values at time points B and D due to fatty liver seen in ultrasonography. Elevated levels of SGPT at time point A and SGOT at time point B were observed in one patient without any clinical and radiological relevance. Three patients had increased levels of SGPT at time point A, as a result of hepatotoxicity of chemotherapy drugs in 2 patients and hepatomegaly and fatty liver seen in ultrasonography in another patient. Among them SGPT levels were observed within normal limits till end of the treatment in 2 patients. Third patient had normal level of SGPT at time point B, in-spite of ongoing chemotherapy. One patient who suffered from Herpes Zoster at time point C, had elevated levels of SGOT and CRP. Elevated level of SGOT is correlated with hepatotoxicity of anti-retroviral drugs used as a treatment of Herpes zoster in this patient. Marginal raised values of SGPT at time point D in 2 patients were due to increase in size and uptake in Thyroid nodule as seen in PET CT in one patient and Hepatomegaly and hemangioma in liver in CT scan Abdomen/Pelvis in another patient. It should be noted that increase in SGOT and SGPT levels were minimal in all these patients and none of them developed liver metastasis which is responsible for elevated levels of Liver Function Test (LFT).

### Kidney Function Test (KFT)—

### Serum Creatinine and Serum Urea—

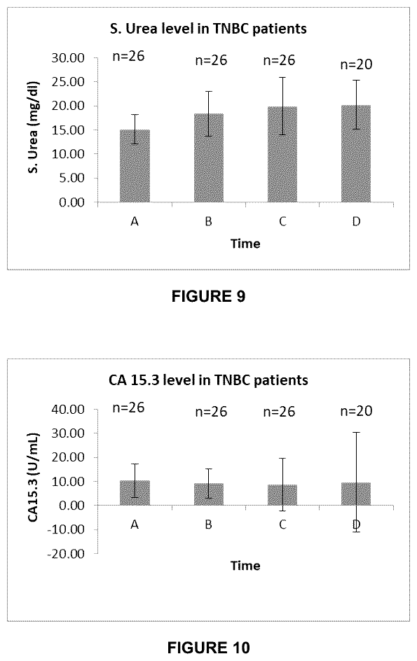

These levels remained within the normal range for all the patients for all four time points (FIGS. 8 and 9).

### Tumor Marker CA 15.3—

Average CA 15.3 level was within normal limits throughout the treatment (FIG. 10). Only in two patients CA 15.3 levels increased at time point C due to metastasis. Among them, there was further increase in CA 15.3 at time point D and another patient expired between time points C and D.

### CRP—

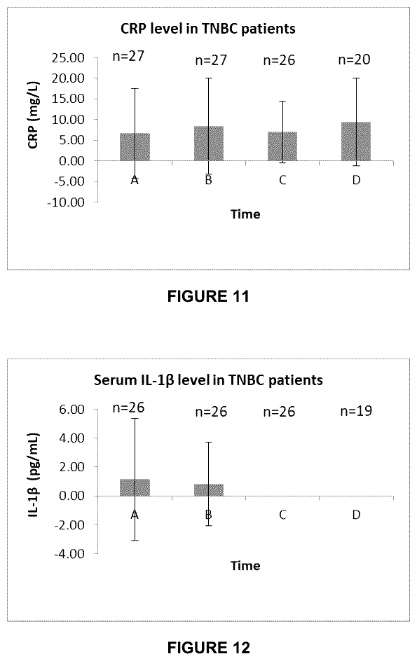

Average CRP levels were stable throughout the treatment (FIG. 11). Few observations regarding clinical and radiological relevance of elevated CRP are noted below. 4 patients had increased levels of CRP at time point A, which subsequently reduced to normal level during complete course of treatment. One patient who had baseline increased CRP level, showed further increase in CRP at time points B and C due to viral fever, stomatitis and herpes zoster, respectively. Two TNBC patients showed elevated CRP levels at time point D and one at time point B due to disease progression.

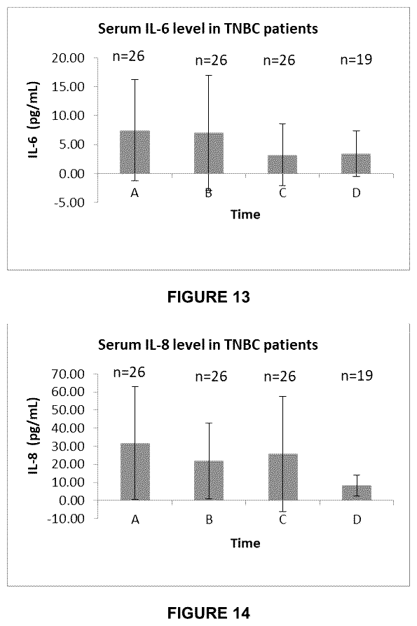

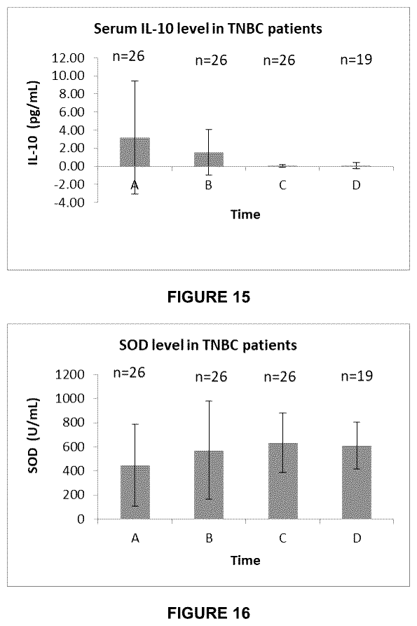

IL1, IL6, IL8 and IL10—Serum levels of cytokines IL-1β, IL-6, IL-8, and IL-10 were assessed in TNBC patients to study the inflammatory response (FIG. 12-15). It is notable that mean serum levels of pro-inflammatory cytokines IL1β, IL6 and IL10 showed progressively decreasing trend at time points B, C and D as compared to baseline levels and were limited to the normal range indicating reduced inflammatory activity. Serum level of anti-inflammatory cytokine, IL-8 was within normal range till time point C and decreased at time point D indicating regulation of immune response upon treatment. These observations indicated immunomodulation in TNBC patients on treatment with the herbo-mineral metallic pharmaceutical kit of the present disclosure.

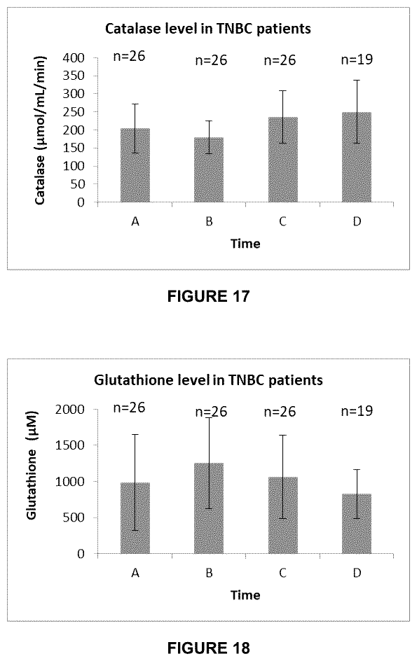

SOD, Catalase and Glutathione—Enzyme (Superoxide dismutase and Catalase) and non-enzyme (Glutathione) antioxidant levels in erythrocyte lysates of TNBC patients were estimated to study the antioxidant effect of Ayurvedic compositions in TNBC patients (FIGS. 16, 17 and 18). The mean SOD activity was within normal range but was higher till time point C which decreased at time point D. On the other hand, mean catalase activity was within normal limits but increased till time point D. Non enzymatic Glutathione levels in these patients revealed decrease at time points C and D. These results depicted enzymatic and non-enzymatic control of redox balance and reduction in oxidative stress based damage in TNBC patients on treatment with the present regime.

### Experiment 2.2: Assessment of Quality of Life (QoL) Using Herbo-Mineral Metallic Pharmaceutical Kit

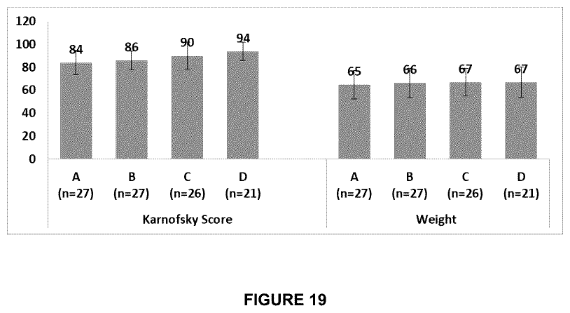

Karnofsky score and weight—The absolute values of Karnofsky score and weight have been represented in the form of graphs of mean values at each time point (FIG. 19). Both showed increasing trend indicating improved performance status of the patients upon treatment with herbo-mineral metallic pharmaceutical kit for the period of 1, 2 and 3 years, respectively. Much higher number of patients indicating positive increase or stable score than the patients with negative increase (decrease) in the Karnofsky score at the end of time points C and D, and overall improved scores indicate long-term effectiveness of treatment with the present herbo-mineral metallic pharmaceutical kit (Table 9, FIG. 19).

TABLE 9
Change in Karnofsky score at time-points B, C, D in
Triple Negative Breast cancer patients treated with the
herbo-mineral metallic pharmaceutical kit.

| | No. of | No. of | No. of |
|---|---|---|---|
| | patients | patients | patients |
| Change in Karnofskyscore | at time | at time | at time |
| compared to time point A | point B | point C | point D |
| Karnofsky score increased | 10 | 13 | 12 |
| Karnofsky score stable | 7 | 7 | 5 |
| Karnofsky score decreased | 10 | 4 | 1 |

Karnofsky score is used to assess well-being of patient. Increase in Karnofsky sore or stable Karnofsky score during the course of treatment is indicative of effectiveness of treatment, whereas decreased Karnofsky score indicates ineffectiveness of treatment due to hampered Quality of Life. The above table depicts number of patients in which Karnofsky score is increased, remained stable or decreased at time points B, C and D. The data indicates effectiveness of Ayurvedic treatment in TNBC patients as Karnofsky score is increased or remained stable in more number of TNBC patients as compared to decreased Karnofsky score at time points B, C and D i.e. at the end of year 1, 2 and 3.

FIG. 19 shows mean of Karnofsky scores and weight at time points A, B, C and D. Increase in mean of Karnofsky score from 80 to 94 and weight from 65 kg to 67 kg is indicative of effectiveness of Ayurvedic treatment in improving well-being during the course of treatment.

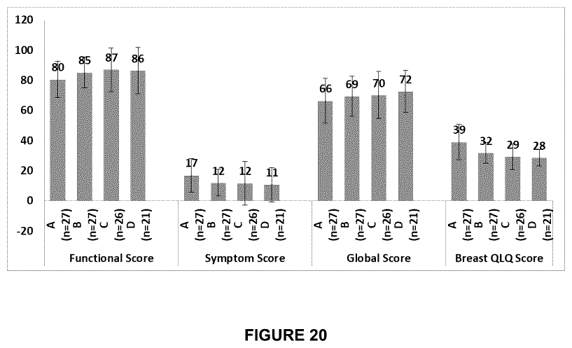

FIG. 20 depicts 4 components of Quality of Life (Derived from QLQ C30 and QLQ BR23) namely Functional score, Global score, Symptom score and Breast score. Increase in Functional and Global score during the course of treatment and decrease in Symptom and Breast score in our study are indicative of effectiveness of Ayurvedic treatment.

QLQ scores—Functional score and global score revealed increasing trend while symptom and breast QLQ score showed decreasing trends from time points A through D suggesting decreased disease load and improved quality of life in these patients (FIG. 20).

Experiment 2.3: Assessment of Cancer Status with Radiological Investigations (PET CT) in TNBC Patients Treated with the Components Inside Herbo-Mineral Metallic Pharmaceutical Kit

TABLE 10
Radiological assessment of
Triple Negative Breast Cancer Patients

| | No. of patients | | | |
|---|---|---|---|---|
| | Before | | | |
| | starting | End of | End of | End of |
| | Treatment | year 1 | year 2 | year 3 |
| | (Time | (Time | (Time | (Time |
| Disease | point | point | point | point |
| Status | A) | B) | C) | D) |
| Disease Free | 26 | 23 | 21 | 18 |
| Survival (DFS) | | | | |
| Overall Survival | 1 | 4 | 5 | 2 |
| (OS) | | | | |
| Expired | 0 | 0 | 1 | 3 at Yr. 3 |
| | | | | 1 at Yr. 2 |
| Yet to complete | 0 | 0 | 0 | 3 |
| three years of | | | | |
| treatment | | | | |
| Total | 27 | 27 | 27 | 27 |

A remarkable finding as seen from Table 10 is that out of 27 patients, 20 patients (74%) survived beyond three years and only four (15%) expired at the end of three years. 18 patients (89%) among 20 had Disease Free Survival (DFS) at the end of year three.

At the end of year 2, 21 patients (78%) among 27 had Disease Free Survival (DFS), whereas 5 patients (18%) had Overall Survival (OS). It is notable that only one TNBC patient expired till end of year two. Out of 27 patients, 23 patients had Disease Free Survival (DFS) at the end of year one. Four patient had Overall Survival (OS) at that time point.

At the same time, it is remarkable that Quality of Life of most of the patients having DFS and OS improved with the treatment with herbo-mineral metallic pharmaceutical kit. Also hematological and biochemical parameters remain in normal limit during the course of treatment. Improvement in immunological parameters and reduction in oxidative stress are attributed to improvement in Quality of Life and manifestation of disease free survival in majority of TNBC patients.

### Experiment 2.4: Data Showing Effect of Components of the Herbo-Mineral Metallic Pharmaceutical Kit on the Survival Pattern in TNBC Patients

A study was carried out in TNBC patients divided in two groups. All patients were started with herbo-mineral-metallic compositions immediately after diagnosis of TNBC. Patients of both the groups were treated with 4 herbo-mineral-metallic compositions, out of which 2 compositions administered to both the groups were similar having similar dosage, whereas 2 compositions were completely different in both the groups. Group one patients were treated with kit of 4 compositions of present disclosure. Patients were treated for a period of three years and assessed for their survival pattern, at time intervals of one year.

TABLE 11
Composition of herbo-mineral metallic compositions
used to determine survival pattern in TNBC patients

| Group 1 (Herbo-mineral | | | Group 2 (Different combination of | | |
|---|---|---|---|---|---|
| metallic pharmaceutical kit composition) | | | herbo-mineral metallic composition) | | |
| Morning - | Suvarna | 2 tablets | Morning - | Suvarna | 2 tablets |
| Evening | Bhasma | (395 mg) | Evening | Bhasmadi Vati | (790 mg) |
| | di Vati | | | | |
| Morning- | Mouktikyukta | 2 tablets | Morning- | Mouktikyukta | 2 tablets |
| Evening | Kamdudha | (500 mg) | Evening | Kamdudha | (500 mg) |
| | Vati | | | Vati | |
| Morning- | Shatavari | 2 tablets | — | — | — |
| Evening | Vati | (500 mg) | | | |
| Before | Arogyavardhini | 2 tablets | After | Padmakadi | 1 tsp |
| lunch and | | (500 mg) | lunch and | Ghruta | (10 gm) |
| dinner | | | dinner | | |
| After lunch | Triphala | 2 tablets | After | Ananta Vati | 4 tablets |
| and dinner | Guggulu | (500 mg) | lunch and | | (1 gm) |
| | | | dinner | | |
| At bedtime | Jatamansi | 10 ml | | | |
| | Oil | | | | |
| | Shiropichu | | | | |

### Time Points for the Above Dosage Administration is as Follows:

A=Baseline
B=End of year 1
C=End of year 2
D=End of year 3

TABLE 12
Observations on survival pattern in TNBC patients

| | Group 1 (n = 27) | | Group 2 (n = 14) | |
|---|---|---|---|---|
| Time points | A | B | A | B |
| DFS | 26 (96%) | 23 (88%) | 14 (100%) | 9 (64%) |
| OS | 1 (4%) | 4 (15%) | 0 | 3 (22%) |
| Expired | 0 | 0 | 0 | 1 (7%) |
| Lost for FU | 0 | 0 | 0 | 1 (7%) |
| Yet to reach | 0 | 0 | 0 | 0 |
| 3 years | | | | |
| Total | 27 | 27 | 14 | 14 |

Triple Negative Breast Cancer (TNBC) is an aggressive type of breast cancer, which has maximum chances of recurrence and/or metastasis within a period of one year after diagnosis. Data shows that 88% TNBC patients of group one i.e. treated with herbo-metallic mineral composition of the present disclosure had Disease Free Survival (DFS), whereas 64% TNBC patients of group 2 had DFS at the end of year one. However, only 15% patients of group one had Overall Survival (OS) against 22% patients of group two had OS at the end of year one. One patient of group two expired due to cancer within a period of one year, whereas all patients in group one survived at the end of year one.

These observations on survival pattern definitely indicate that different combination of herbo-mineral-metallic composition, other than present disclosure does not provide desirable effect in TNBC patients.

### Technical Advancements & Economic Significance

The present disclosure described herein above has several technical advantages including, but not limited to, the realization of a herbo-mineral metallic pharmaceutical kit that:
provides a disease-free survival in TNBC patients; and
improve the quality of life in TNBC patients

The economy significance details requirement may be called during the examination. Only after filing of this patent application, the applicant can work publically related to present disclosure product/process/method. The applicant will disclose all the details related to the economic significance contribution after the protection of invention.

The embodiments as described herein above, and various features and advantageous details thereof are explained with reference to the non-limiting embodiments in the description.

Descriptions of well-known aspects, components, and molecular biology techniques are omitted so as to not unnecessarily obscure the embodiments herein.

The foregoing description of specific embodiments so fully reveal the general nature of the embodiments herein, that others can, by applying current knowledge, readily modify and/or adapt for various applications of such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein. Further, it is to be distinctly understood that the foregoing descriptive matter is to be interpreted merely as illustrative of the disclosure and not as a limitation.

Having described and illustrated the principles of the present disclosure with reference to the described embodiments, it will be recognized that the described embodiments can be modified in arrangement and detail without departing from the scope of such principles.

While considerable emphasis has been placed herein on the particular features of this disclosure, it will be appreciated that various modifications can be made, and that many changes can be made in the preferred embodiment without departing from the principles of the disclosure. These and other modifications in the nature of the disclosure or the preferred embodiments will be apparent to those skilled in the art from the disclosure herein, whereby it is to be distinctly understood that the foregoing descriptive matter is to be interpreted merely as illustrative of the disclosure and not as a limitation.

1-11. (canceled)

12. A herbo-mineral metallic pharmaceutical kit for delaying/controlling recurrence and/or metastasis of cancer in Triple Negative Breast Cancer (TNBC) patients, said kit comprising:
i. a first container containing Suvarna Bhasmadi Vati (SBV) in a solid dosage form, in an amount in the range of 250 mg/day to 450 mg/day;
ii. a second container containing Mouktikyukta Kamdudha Vati (MKV) in a solid dosage form, in an amount in the range of 300 mg/day to 600 mg/day;
iii. a third container containing Shatavari Vati (SV) in a solid dosage form, in an amount in the range of 300 mg/day to 600 mg/day;
iv. a fourth container containing Arogyavardhini Vati (AVV) in a solid dosage form, in an amount in the range of 300 mg/day to 600 mg/day;
v. a fifth container containing Triphala Guggul Vati (TGV) in a solid dosage form, in an amount in the range of 300 mg/day to 600 mg/day.
vi. a sixth container containing jatamansi Oil (JMO) in the form of mediated oil for external application in an amount in the range of 5 ml/day to 15 ml/day.

13. The kit as claimed in claim 12, wherein said Suvarna Bhasmadi Vati (SBV) is prepared from:
Suvarna bhasma in an amount ranging from 2 wt. % to 7 wt. % of the total weight of said SBV, wherein the particle size of suvarna bhasma is in the range of 200 nm to 500 nm;
Mouktik bhasma in an amount ranging from 20 wt. % to 35 wt. % of the total weight of said SBV, wherein the particle size of mouktik bhasma is in the range of 55 μm to 73 μm;
Guduchi sattva in an amount ranging from 45 wt. % to 60 wt. % of the total weight of said SBV; and
at least one excipient in an amount ranging from 5 wt. % to 30 wt. % of the total weight of said SBV.

14. The kit as claimed in claim 12, wherein said Mouktikyukta Kamdudha Vati (MKV) is prepared from:
Mouktik bhasma in an amount ranging from 10 wt. % to 14 wt. % of the total weight of said MKV;
Shankha bhasma in an amount ranging from 10 wt. % to 14 wt. % of the total weight of said MKV;
Shouktik bhasma in an amount ranging from 10 wt. % to 14 wt. % of the total weight of said MKV;
Kapardik bhasma in an amount ranging from 10 wt. % to 14 wt. % of the total weight of said MKV;
Praval bhasma in an amount ranging from 10 wt. % to 14 wt. % of the total weight of said MKV;
Guduchi sattva in an amount ranging from 10 wt. % to 14 wt. % of the total weight of said MKV;
Shudhha Gairik in an amount ranging from 10 wt. % to 14 wt. % of the total weight of said MKV; and
at least one excipient in an amount ranging from 10 wt. % to 30 wt. % of the total weight of said MKV.

15. The kit as claimed in claim 12, wherein said Shatavari Vati (SV) is prepared from:
Shatavari powder in an amount ranging from 75 wt. % to 92 wt. % of the total weight of said SV wherein the powder has a particle size in the range of 150 to 180 microns, and
at least one excipient in an amount ranging from 8 wt. % to 25 wt. % of the total weight of said SV.

16. The kit as claimed in claim 12, wherein said Arogyavardhini Vati (AVV) is prepared from:
Kajjali in an amount ranging from 2.3 wt. % to 2.5 wt. % of the total weight of said AVV, wherein the particle size of kajjali is in the range of 53 μm to 73 μm;
loha bhasma in an amount ranging from 1.1 wt. % to 1.3 wt. % of the total weight of said AVV, wherein the particle size of loha bhasma is in the range of 53 μm to 73 μm;
Abhrak bhasma in an amount ranging from 1.1 wt. % to 1.3 wt. % of the total weight of said AVV, wherein the particle size of abhrak bhasma is in the range of 53 μm to 73 μm;
Tamra bhasma in an amount ranging from 1.1 wt. % to 1.3 wt. % of the total weight of said AVV, wherein the particle size of tamara bhasma is in the range of 53 μm to 73 μm;
Haritaki powder in an amount ranging from 2.3 wt. % to 2.5 wt. % of the total weight of said AVV, wherein the particle size of haritaki is in the range of 150 μm to 180 μm;
Bibhitaki powder in an amount ranging from 2.3 wt. % to 2.5 wt. % of the total weight of said AVV, wherein the particle size of bibhitaki is in the range of 150 μm to 180 μm;
Amalaki powder in an amount ranging from 2.3 wt. % to 2.5 wt. % of the total weight of said AVV, wherein the particle size of amalaki is in the range of 150 μm to 180 μm;
Shuddha Shilajit in an amount ranging from 3.5 wt. % to 3.7 wt. % of the total weight of said AVV, wherein the particle size of Shuddha Shilajit is in the range of 355 μm to 450 μm;
Shuddha Guggul in an amount ranging from 4.7 wt. % to 4.9 wt. % of the total weight of said AVV, wherein the particle size of Shuddha Guggul is in the range of 355 μm to 450 μm;
Chitrak powder in an amount ranging from 4.7 wt. % to 4.9 wt. % of the total weight of said AVV, wherein the particle size of chitrak is in the range of 150 μm to 180 μm;
Kutaki powder in an amount ranging from 26.2 wt. % to 26.8 wt. % of the total weight of said AVV, wherein the particle size of kutaki is in the range of 150 μm to 180 μm;
the dry extract from Nimba leaf juice in an amount ranging from 22 wt. % to 22.4 wt. % of the total weight of said AVV; and
at least one excipient in an amount ranging from 23.4 wt. % to 26.4 wt. % of the total weight of said AVV.

17. The kit as claimed in claim 12, wherein said Triphala Guggul Vati (TGV) is prepared from:
Amalaki powder in an amount ranging from 9.6 wt. % to 11.6 wt. % of the total weight of said TGV;
Bibhitaki powder in an amount ranging from 9.6 wt. % to 11.6 wt. % of the total weight of said TGV;
Haritaki powder in an amount ranging from 9.6 wt. % to 11.6 wt. % of the total weight of said TGV;
Pippali powder in an amount ranging from 9.6 wt. % to 11.6 wt. % of the total weight of said TGV;
Shuddha Guggul in an amount ranging from 50 wt. % to 56 wt. % of the total weight of said TGV; and
at least one gliding agent in an amount ranging from 3.6 wt. % to 5.6 wt. % of the total weight of said TGV.

18. The kit as claimed in claim 12, wherein said jatamansi Oil (JMO) is prepared from:
Extract of dried rhizomes of jatamansi in an amount ranging from 0.5 wt. % to 98 wt. % of the total weight of said JMO; and
Til taila/sesame oil as base in an amount ranging from 99.5 wt. % to 98 wt. % of the total weight of said JMO.

19. The kit as claimed in claim 12, wherein said solid dosage form of SBV, MKV, SV, AVV and TGV is selected from the group consisting of tablet, pill, and capsule.

20. The kit as claimed in claim 13, wherein said excipient is a binder.

21. The kit as claimed in claim 14, wherein said excipient is a binder.

22. The kit as claimed in claim 15, wherein said excipient is a binder.

23. The kit as claims in claim 16, wherein said excipient is a binder.

24. The kit as claimed in claim 20, wherein said binder is selected from the group consisting of gum acacia, guar gum and xanthan gum.

25. The kit as claimed in claim 21, wherein said binder is selected from the group consisting of gum acacia, guar gum and xanthan gum.

26. The kit as claimed in claim 22, wherein said binder is selected from the group consisting of gum acacia, guar gum and xanthan gum.

27. The kit as claimed in claim 23, wherein said binder is selected from the group consisting of gum acacia, guar gum and xanthan gum.

28. The kit as claimed in claim 17, wherein said gliding agent is talc.
